(19) United States
(12) Patent Application Publication
(10) Pub. No.: US 2025/0215462 A1
(43) Pub. Date: Jul. 3, 2025

(54) SINGLE CELL PROTEIN WITH ENHANCED POLYUNSATURATED FATTY ACID CONTENT

(71) Applicant: LanzaTech, Inc., Skokie, IL (US)

(72) Inventors: Sean Simpson, Auckland (NZ); Timothy Politano, Chicago, IL (US); James Clomburg, Chicago, IL (US); Alex Chou, Lincolnwood, IL (US); Scott Kleespies, Chicago, IL (US)

(21) Appl. No.: 18/976,903

(22) Filed: Dec. 11, 2024

Related U.S. Application Data
(60) Provisional application No. 63/608,784, filed on Dec. 11, 2023.

(51) Int. Cl. C12P 7/6427; C12N 9/02

(52) U.S. Cl. CPC C12P 7/6427; C12N 9/0071; C12Y 114/19

(57) ABSTRACT

The present disclosure relates to single cell protein (SCP) products obtained from Cupriavidus necator that has been engineered to produce a favorable fatty acid profile. The biomass produced by the C. necator and the SCP products that include it contain unsaturated fatty acids that are not naturally produced by C. necator.

### CROSS REFERENCE TO RELATED APPLICATIONS

This application claims the benefit of U.S. Provisional Patent Application No. 63/608,784 filed on Dec. 11, 2023, the entirety of which is incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on Nov. 12, 2024, is named LT293US1-Sequences.xml and is 7,384,290 bytes in size.

### BACKGROUND

The following discussion is merely provided to aid the reader in understanding the disclosure and is not admitted to describe or constitute prior art thereto.

Cupriavidus necator is a type of bacteria commonly used for the production of single cell protein for food and feed applications. It is generally high in protein content with a favorable amino acid profile. However, the fatty acid profile of C. necator is intrinsically low in nutritionally valuable polyunsaturated fatty acids, including omega-3 fatty acids.

### SUMMARY

The present disclosure provides nutritionally enhanced single cell protein (SCP) with high polyunsaturated fatty acid content. Specifically, the present disclosure demonstrates the ability to tailor the fatty acid profile of C. necator toward unsaturated C18 fatty acids, including C18:2 and C18:3 compounds, as well as unsaturated C16 fatty acids.

In one aspect, the present disclosure provides strains of Cupriavidus necator, comprising a transgene encoding a heterologous desaturase enzyme, wherein the strain of C. necator synthesizes at least one unsaturated C18 fatty acid that is not natively produced by C. necator. The at least one unsaturated C18 fatty acid may be selected from an oleic acid, a linoleic acid, an α-linoleic acid, a γ-linolenic acid, a linolelaidic acid, an elaidic acid, a vaccenic acid, a stearidonic acid, and any combination thereof.

In another aspect, the present disclosure provides strains of C. necator that expresses a heterologous desaturase enzyme encoded by a transgene, wherein the strain of C. necator synthesizes an increased amount of an unsaturated C1 and/or C18 fatty acid relative to a strain of C. necator that does not express the heterologous desaturase enzyme. The unsaturated fatty acid may be a C16 fatty acid, a C18 fatty acid, or both a C16 fatty acid and a C18 fatty acid. More specifically, the C16 fatty acid may be selected from a palmitoleic acid, a sapienic acid, and any combination thereof. Similarly, the C18 fatty acid may be selected from an oleic acid, a linoleic acid, an α-linoleic acid, a γ-linolenic acid, a linolelaidic acid, an elaidic acid, a vaccenic acid, a stearidonic acid, and any combination thereof.

The transgene(s) used for the purposes of any of the disclosed aspects or embodiments may be codon optimized for expression in Cupriavidus necator.

For the purposes of the present disclosure, the strain of C. necator may be modified by, for example, adaptive laboratory evolution or genetic modification.

For the purposes of the present disclosure, the strain of C. necator may be a strain that grows autotrophically at up to 40° C. For example, the strain of C. necator may be the strain deposited at DSM34774. Additionally or alternatively, the strain of C. necator may have a H₂:CO₂ uptake ratio that is lower than the H₂:CO₂ uptake ratio of a wild type or naturally occurring strain of C. necator cultured under corresponding temperatures and conditions. In some embodiments, the strain of C. necator may comprise a full or partial deletion of a gene or locus encoding a membrane-bound hydrogenase, such as hoxKGXZ.

The strain of C. necator may also be a strain that produces high levels of protein, which has a favorable amino acid profile. For example, the strain of C. necator may produce about 40%, about 50%, about 60%, about 70%, about 80%, about 90%, or about 95% by weight of protein.

The heterologous desaturase enzyme(s) expressed by the disclosed strains of C. necator may be selected from a Δ9-desaturase, a Δ12-desaturase, a Δ15-desaturase, a Δ6-desaturase, a Δ5-desaturase, and a Δ4-desaturase. Such heterologous desaturase enzymes can be from a cyanobacteria, such as a Synechococcus species (sp), or a eukaryote, such as a yeast.

For the purposes of the present disclosure, the strain of C. necator may further comprise a second transgene encoding a second heterologous desaturase enzyme, including, but not limited to, a Δ9-desaturase, a Δ12-desaturase, a Δ15-desaturase, a Δ6-desaturase, a Δ5-desaturase, and a Δ4-desaturase. The second heterologous desaturase enzyme can be from a cyanobacteria, such as a Synechococcus species (sp), or a eukaryote, such as a yeast. The strain of C. necator may further comprise a third transgene encoding a third heterologous desaturase enzyme, including, but not limited to, a Δ9-desaturase, a Δ12-desaturase, a Δ15-desaturase, a Δ6-desaturase, a Δ5-desaturase, and a Δ4-desaturase. The third heterologous desaturase enzyme can be from a cyanobacteria, such as a Synechococcus species (sp), or a eukaryote, such as a yeast.

For the purposes of the present disclosure, the strain of C. necator may express a second heterologous desaturase enzyme, including, but not limited to, a Δ9-desaturase, a Δ12-desaturase, a Δ15-desaturase, a Δ6-desaturase, a Δ5-desaturase, and a Δ4-desaturase. The second heterologous desaturase enzyme can be from a cyanobacteria, such as a Synechococcus species (sp), or a eukaryote, such as a yeast. The strain of C. necator may further express a third heterologous desaturase enzyme, including, but not limited to, a Δ9-desaturase, a Δ12-desaturase, a Δ15-desaturase, a Δ6-desaturase, a Δ5-desaturase, and a Δ4-desaturase. The third heterologous desaturase enzyme can be from a cyanobacteria, such as a Synechococcus species (sp), or a eukaryote, such as a yeast.

For the purposes of the present disclosure, the strain of C. necator may express at least one of DesC, DesA, or DesB from a Synechococcus sp. Alternatively, the strain of C. necator may express all of DesC, DesA, and DesB from a Synechococcus sp.

For the purposes of the present disclosure, the strain of C. necator may further comprise or express a transgene encoding an enzyme to increase C18:0 fatty acid synthesis. For example, the strain of C. necator may express or overexpresses at least one of FabD, FabB, or FabF. Alternatively, the strain of C. necator may express or overexpresses FabD, FabB, and FabF. The strain of C. necator may express or overexpresses at least one of FabG or FabZ, or the strain of C. necator may express or overexpresses both FabG and FabZ. In some embodiments, C18:0 fatty acid synthesis may be increased at least 50% relative to a wild-type strain of C. necator.

In another aspect, the present disclosure provides methods of preparing a single-cell protein product, comprising:
(a) culturing a strain of C. necator disclosed herein (e.g., any of the foregoing aspects or embodiments), thereby creating biomass comprising protein and fatty acid,
(b) isolating the C. necator or the biomass, and
(c) preparing a single-cell protein product from the C. necator or the biomass.

In another aspect, the present disclosure provides methods of producing a fatty acid, comprising culturing a strain of C. necator disclosed herein (e.g., any of the foregoing aspects or embodiments) such that the fatty acid is produced. In some embodiments, the methods may further comprise isolating the fatty acid or isolating the strain of C. necator.

Culturing may comprise growing the strain of C. necator in a fermentation tank. The fermentation tank can be a gas fermentation tank.

Culturing may also comprise growing the strain of C. necator in autotrophic conditions. The autotrophic conditions comprise providing carbon monoxide (CO) or carbon dioxide (CO₂) as a carbon source. Hydrogen gas may also be present.

Culturing may also comprise growing the strain of C. necator at a temperature above 30° C. and up to 40° C. For example, the temperature of the culture may be 31° C., 32° C., 33° C., 34° C., 35° C., 36° C., 37° C., 38° C., 39° C., or 40° C.

In another aspect, the present disclosure provides single-cell protein (SCP) compositions, comprising a strain of C. necator disclosed herein (e.g., any of the foregoing aspects or embodiments) or biomass created therefrom.

The composition may comprise at least about 40%, about 50%, about 60%, about 70%, about 80%, about 90%, or about 95% by weight of protein.

The composition may comprise at least about 0.1%, about 0.5%, about 1%, about 2%, about 5%, about 10% or about 15% of a fatty acid component. The fatty acid component may comprise an oleic acid, a linoleic acid, an α-linoleic acid, a γ-linolenic acid, a linolelaidic acid, an elaidic acid, a vaccenic acid, a stearidonic acid, or any combination thereof.

The composition can be produced by culturing a strain of C. necator disclosed herein, thereby creating biomass comprising protein and fatty acid, and isolating the C. necator or the biomass.

In another aspect, the present disclosure provides edible products, comprising (a) a strain of C. necator disclosed herein or biomass created therefrom, or (b) an SCP composition disclosed herein.

The edible product may be an animal feed, such as a fish feed, a livestock or ruminant feed, a swine feed, goat feed, llama feed, turkey feed, a poultry feed, a rodent feed, a dog feed, and a cat feed.

The edible product may be a human food, such as a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

In another aspect, the present disclosure provides method of preparing an edible product, comprising mixing (a) a strain of C. necator disclosed herein or biomass created therefrom, or (b) an SCP composition of C. necator disclosed herein with one or more edible ingredients.

In another aspect, the present disclosure provides topical product, comprising a fatty acid produced by a method disclosed herein or from a strain of C. necator disclosed herein. The topical product may be, for example, a lotion, balm, cream, or makeup.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF DRAWINGS

These and other aspects of the present disclosure, which should be considered in all its novel aspects, will become apparent from the following description, which is given by way of example only, with reference to the accompanying figures, in which:

FIG. 1 (FIG. 1): shows biomass titer (OD600) and reactor temperature throughout the ˜100 day temperature-tolerance evolution of DSM 541 in CSTR. Reactor inputs were kept consistent throughout the experiment, while reactor temperature was gradually increased step-wise.

FIG. 2 (FIG. 2): shows biomass titer (OD600) and reactor temperature of a representative autotrophic CSTR run with DSM 34774. Reactor temperature was held constant at 37° C. demonstrating the ability of the temperature evolved strain to grow on CO₂/H₂/O₂ gas mixtures at elevated temperatures.

FIGS. 3A-3B (FIGS. 3A-3B): show biosynthesis of polyunsaturated fatty acids. FIG. 3A demonstrates native fatty acid biosynthesis pathway in C. necator with FabF catalyzing the initial chain elongation step. FIG. 3B demonstrates a heterologous pathway for biosynthesis of non-native unsaturated fatty acids in Cupriavidus necator.

FIG. 4 (FIG. 4): shows a fatty acid profile of continuously CO₂/H₂ gas grown C. necator. A GC-MS fatty acid profile of gas-grown C. necator indicating predominately C16 fatty acid profile of both C16:0 and C16:1 cis-49. Also indicated by GC-MS are trace amounts of myristic acid (C14:0), C12:0 and C18:0 fatty acids. C18:1 peaks indicated C18 unsaturated fatty acids in addition to oleic and elaidic acids that dictate pathways for producing ω-3 fatty acids. GC-MS results also indicates trace amounts of C17 cyclopropane fatty acids.

FIG. 5 (FIG. 5): shows an unknown peak corresponding to C18:1 fatty acid generated by gas grown C. necator indicates the production of cis-vaccenic acid. A close-up image of the GC-MS results from 17:83-20.92 minutes of FIG. 6 indicating an unknown peak at 19:50 minutes is possibly cis-vaccenic acid (18:1). Cis-vaccenic acid standard controls are indicated in red. Overlapping peaks support the hypothesis that gas-grown C. necator synthesized C18:1 cis-vaccenic acid.

FIG. 6 (FIG. 6): shows oleic acid biosynthesis demonstrated by expression of DesC (delta-9 desaturase) from Synechococcus sp. PCC 7002. FIG. 6 shows GC-MS analysis of the C18 region for heterologous DesC expressing C. necator. Empty vector controls are indicated in black. GC-MS indicates the presence of oleic acid (C18:1 cis Δ9) in heterologous DesC expressing C. necator but not in empty vector controls. Also shown are peaks for cis-vaccenic acid (C18:1Δ11) and stearic acid (C18:0), indicating a 50% conversion of C18:0 to C18:149 as compared to empty vector controls.

FIGS. 7A-7B (FIGS. 7A-7B): show FabF overexpression substantially improves oleic acid production. GC-MS analysis indicating overexpression of C. necator FabF with DesC (FIG. 7A; blue) increases observed oleic acid production in comparison to DesC alone (FIG. 7A; black). Cells expressing FabF enzyme demonstrate increased biomass and cell growth after 48 hours. Optical density measurements indicate decreased optical density in cells expressing FabF enzyme indicating increased cell growth and polyunsaturated fatty acid synthesis (FIG. 7B).

FIGS. 8A-8D (FIGS. 8A-8D): show linolenate peak is observed when CnFabF is overexpressed along with DesA DesB and DesC (deslta-9, delta-12, and omega-3 desaturases, respectively). GCMS of methyl linolenate standard controls demonstrate observable peaks at 19.3 minutes (FIG. 8A). FIG. 8B shows GC-MS of C. necator overexpressing CnFabF in combination with DesABC expression indicating possible linolenate biosynthesis as confirmed by overlapping peak of methyl linolenate standard control (FIG. 8A) at 19.3 minutes. The combination of CnFabF and DesC is required for linolenate biosynthesis and corresponding GC-MS peaks. CnFabF+DesAB (FIG. 8C) and DesABC alone do not present corresponding linolenate peaks. DesABC expression does not present a corresponding linolenate peak, indicating the FabF expression is required for linolenate biosynthesis (FIG. 8D).

FIG. 9 (FIG. 9): shows a map of plasmid pACC30.1 used for the production of linolenic acid in C. necator.

FIGS. 10A-10C (FIGS. 10A-10C): show open reading frames (ORFs) in the Δ35433 bp encompassing genes [E6A55_33395]-[E6A55_33560] of the C. necator genome.

### DETAILED DESCRIPTION

The disclosure provides engineered Cupriavidus necator bacteria that express at least one desaturase enzyme such that the bacteria synthesizes at least one unsaturated C18 fatty acid that is not natively produced by C. necator. The bacteria may be a novel strain of heat-adapted C. necator bacteria.

### I. DEFINITIONS

It is to be understood that the disclosed compositions and methods are not limited to the particular implementations described, and as such may vary. It is also to be understood that the terminology used herein is for the purpose of describing particular implementations only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “about” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11”.

As used herein, the term “bioproduction” is intended to mean production of a compound by way of biological or enzymatic synthesis (as opposed to chemical synthesis). In some implementations, bioproduction may be performed by a transgenic organism or microbe (e.g., C. necator) that has been engineered to express enzymes involved in the biological synthesis of a compound of interest (e.g., a fatty acid).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “protein” is a biological macromolecule comprised of one or more chain(s) of amino acids. An “enzyme” is a type of protein that possesses a biological catalytic activity that accelerates chemical reaction. Thus, for the purposes of this disclosure, enzymes are an example of a protein that can catalyze a reaction, such as the production of a particular fatty acid.

The terms “engineered cell” or “engineered host cell” refer to a modified cell wherein the modification can be selected from e.g., increased expression of a gene, inhibited expression of a gene, knockout of a gene or genes, introduction of a new gene or genes, introduction of mutant gene(s), or mutation/genetic alteration of gene(s), wherein the increased expression or inhibited expression of a gene can be achieved by using techniques, such as gene deletion, changed gene copy number, changed gene promoter (e.g. by using a strong or weak promoter), etc. An engineered cell or engineered host cell may also include a cell that has been isolated. In some implementations, an engineered cell or engineered host cell is a transgenic cell. In some implementations, an engineered cell or engineered host cell is a transgenic cell capable of producing high levels of a compound or biomolecule of interest. An example of a host cell herein may be a C. necator cell.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The terms “derived from” or “derivative” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes.

For the purposes of this disclosure, all of the compounds (e.g., fatty acids), enzymes, and cells disclosed herein can be isolated in a form that is substantially free of other proteins, contaminants, macromolecules (e.g., nucleic acids, lipids, etc.), or cells. However, it should be understood that an “isolated” fatty acid or enzyme may not be 100% free of other proteins, contaminants, or macromolecules, and absolute purity is not required in order for a protein or enzyme to be considered “isolated.”

For the purposes of this disclosure, a “wild type” is the phenotype or sequence of the typical form of a cell, protein, or enzyme as it occurs in nature (i.e., the “normal” or “standard” cell or protein sequence, as opposed to an engineered or otherwise altered variant or a naturally occurring mutant).

As used herein, “optional” or “optionally” means that the subsequently described event or circumstance may or may not occur, and that the description includes instances where said event or circumstance occurs and instances where it does not.

The term “metabolic trait” refers to metabolic features of the cell or organisms, e.g. the capability to produce a certain product under certain conditions, the capability to survive and reproduce under certain conditions, such as certain pH, temperature, certain gas levels, or certain nutrients, preferably certain temperatures or certain nutrients.

As used herein, the term “vector” refers to a nucleic acid molecule that can transport another nucleic acid to which it is attached. One type of vector is a “plasmid,” which stands for a circular double-stranded DNA loop into which additional DNA segments can be ligated. Another type of vector is a viral vector, whereby additional DNA segments can be ligated into the viral genome. Certain vectors can replicate autonomously in a host cell into which they have been introduced (e.g., bacterial vectors with a bacterial origin of replication). Other vectors are advantageously integrated into the genome of a host cell when introduced into the host cell and are thereby replicated together with the host genome. In addition, certain vectors can control the expression of genes to which they are operably linked. These vectors are referred to here as “expression vectors.” Usually expression vectors suitable for recombinant DNA techniques are in the form of plasmids. In the present description, “plasmid” and “vector” can be used interchangeably because the plasmid is the most commonly used vector form. However, the disclosure is intended to encompass these other expression vector forms, such as viral vectors, which perform similar functions.

“Transgene” or “recombinant” as used in the present disclosure, for example with respect to a nucleic acid sequence, an expression cassette, gene construct, or a vector containing the nucleic acid sequence according to the disclosure or an organism transformed with the nucleic acid sequences, expression cassette or vector according to the disclosure, all those obtained by genetic engineering methods Constructions in which either a) the nucleic acid sequence according to the disclosure, or b) a genetic control sequence functionally linked to the nucleic acid sequence according to the disclosure, for example a promoter, or c) (a) and (b) are not in their natural, genetic environment or were modified by genetic engineering methods, the modification being an example.

A transgenic organism or transgenic bacteria in the sense of the disclosure is to be understood to mean that the nucleic acids used in the method do not bind their natural position in the genome of an organism, the nucleic acids can be expressed homologously or heterologously. However, as mentioned, transgene also means that the nucleic acids according to the disclosure are in their natural place in the genome of an organism, but that the sequence has been changed compared to the natural sequence and/or that the regulatory sequences of the natural sequences have been changed. Transgenic is preferably to be understood as meaning the expression of the nucleic acids according to the disclosure at a non-natural location in the genome, that is to say that the nucleic acids are homologous or preferably heterologous.

As used herein, the term “autotrophic bacteria” refers to a bacteria that is capable of producing all its biomass carbon from CO₂.

For the purpose of the description, a phrase in the form “A/B” or in the form “A and/or B” means (A), (B), or (A and B).

### II. C1-FIXING MICROORGANISMS

The microorganism of the disclosure may also be derived from essentially any parental microorganism, such as a parental microorganism selected from the group consisting of Escherichia coli and Saccharomyces cerevisiae.

In another embodiment, the microorganism of the disclosure is an aerobic bacterium. In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the strain is Cupriavidus necator DSM 428, DSM 531, or DSM541, or any derivatives thereof. In another embodiment, the strain is Cupriavidus necator DSM 34774. In some embodiments, the strain is SEQ ID. NO: 1. In some embodiments, the strain is a derivative of Cupriavidus necator DSM 34774. In another embodiment, the strain is a derivative of SEQ ID. NO: 1.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta (3)-cis-delta (2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In the microorganisms of the disclosure, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes. In an embodiment, limitation selected from nutrients, dissolved oxygen, or any combination thereof diverts carbon flux to desired products.

In an embodiment, the fermentation broth comprises the feed streams in combination with the aerobic microorganism in the bioreactor. In some embodiments, the feed streams, e.g., a carbon source feed stream, a flammable gas-containing stream, and an oxygen-containing gas feed stream, react with the microorganism in the bioreactor to at least partially form the fermentation broth (which may also include other products, byproducts, and other media fed to the bioreactor). The unreacted oxygen, or the oxygen that is not consumed by the microorganism, exists as both dissolved oxygen and gaseous oxygen in a dispersed gaseous phase within the fermentation broth. The same holds true for the other gases that are soluble. The dispersed gaseous phase, containing the unreacted components, e.g., oxygen, nitrogen, hydrogen, carbon dioxide and/or water vapor, rises to the headspace of the bioreactor.

In some embodiments, an oxygen-containing gas, e.g., air, can be fed directly into the fermentation broth. In one embodiment, the oxygen-containing gas can be an oxygen-enriched source, e.g., oxygen-enriched air or pure oxygen. In an embodiment, the oxygen-containing gas may comprise greater than 6.0 vol. % of oxygen, e.g., greater than 10.0 vol. %, greater than 20.0 vol. %, greater than 40.0 vol. %, greater than 60.0 vol. %, greater than 80.0 vol. %, or greater than 90.0 vol. %. In some embodiments, the oxygen-containing gas may be pure oxygen.

In one embodiment, the microorganism of the disclosure is capable of producing ethylene. One embodiment is directed to a recombinant C1-fixing microorganism capable of producing ethylene from a carbon source comprising a nucleic acid encoding a group of exogenous enzymes comprising at least one ethylene forming enzyme (EFE). In some embodiments the EFE is derived from Pseudomonas syringae. In an embodiment, the EFE has an E.C. number 1.13.12.19. The microorganism of an embodiment comprising at least one EFE having an E.C. number 1.13.12.19. The microorganism of an embodiment, further comprising a nucleic acid encoding a group of exogenous enzymes comprising at least one alpha-ketoglutarate permease (AKGP).

The microorganism of an embodiment, wherein a nucleic acid encoding a group of exogenous enzymes comprises at least one EFE, at least one AKGP, or any combination thereof. The microorganism of an embodiment, wherein a nucleic acid encoding a group of exogenous enzymes comprises at least one EFE and at least one AKGP. The microorganism of an embodiment, wherein the nucleotide encoding a group of exogenous enzymes is inserted into a bacterial vector plasmid, a high copy number bacterial vector plasmid, a bacterial vector plasmid having an inducible promoter, a nucleotide guide of a homologous recombination system, a CRISPR Cas system, or any combination thereof. In an embodiment, the promoter is a phosphate limited inducible promoter. In some embodiments, the promoter is a nitrogen limited promoter. In some embodiments, the promoter is an NtrC-P activated promoter. In some embodiments, the promoter is a H₂ inducible promoter. In one embodiment, the microorganism comprises an intracellular oxygen concentration limit. In another embodiment, the method limits intracellular oxygen concentration. In one embodiment, the method comprises a step of controlling dissolved oxygen. In an embodiment, the method comprises decreased ethylene production with decreased dissolved oxygen concentration. In some embodiments, the microorganism comprises a molecular switch. In some embodiments, the microorganism comprises an ability to switch the cellular burden under variable conditions.

In some embodiments, the microorganism is a natural or an engineered microorganism that is capable of converting a gaseous substrate as a carbon and/or energy source. In one embodiment, the gaseous substrate includes CO₂ as a carbon source. In some embodiments, the gaseous substrate includes H₂, and/or O₂ as an energy source. In one embodiment, the gaseous substrate includes a mixture of gases, comprising H₂ and/or CO₂ and/or CO.

In some embodiments, the gas fermentation product is selected from an alcohol, an acid, a diacid, an alkene, a terpene, an isoprene, and alkyne. In some embodiments, the method and microorganism disclosed herein are for the improved production of ethylene. In an embodiment, the method and microorganism disclosed herein are for the improved production of a gas fermentation product.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1-propanol, 1-hexanol, 1-octanol, a fatty alcohol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monocthylene glycol, or any combination thereof.

The disclosure provides microorganisms capable of producing ethylene comprising culturing the microorganism of the disclosure in the presence of a substrate, whereby the microorganism produces ethylene.

As used herein, the terms “intermediate” and “precursor” can be used interchangeably to refer to a substance, such as a molecule, compound, or protein, that is produced upstream of a particular product. The intermediate may be directly upstream of the product. The intermediate may be indirectly upstream of the product. For example, in the exemplary reaction “compound A” à “compound B” à “compound C” à “compound D”, “compound” C is an intermediate that is directly upstream of the product, “compound D,” and “compound B” is an intermediate that is indirectly upstream of the product, “compound D.”

The enzymes of the disclosure may be codon optimized for expression in the microorganism of the disclosure. “Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the disclosure are codon optimized for expression in the microorganism of the disclosure. Although codon optimization refers to the underlying genetic sequence, codon optimization often results in improved translation and, thus, improved enzyme expression. Accordingly, the enzymes of the disclosure may also be described as being codon optimized.

One or more of the enzymes of the disclosure may be overexpressed. “Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The enzymes of the disclosure may comprise a disruptive mutation. A “disruptive mutation” refers to a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

The disclosed microorganisms, including C. necator, may utilize various carbon sources, including gaseous carbon sources as a substrate.

In some embodiments, the substrate comprises CO₂ and an energy source. In some embodiments, the substrate comprises CO₂ and an energy source. In an embodiment, the substrate comprises CO₂, H₂, and O₂. In some embodiments, the substrate comprises CO₂ and any suitable energy source. In one embodiment, the substrate comprises CO. In one embodiment, the substrate comprises CO₂ and CO. In another embodiment, the substrate comprises CO₂ and H₂. In another embodiment, the substrate comprises CO₂ and CO and H₂.

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14:127, 2015). In some embodiments, the substrate does not comprise a pentose such as xylose (Pereira, Metab Eng, 34:80-87, 2016). In some embodiments, the substrate may comprise both gaseous and carbohydrate substrates (mixotrophic fermentation). The substrate may further comprise other non-carbon components, such as H₂, N₂, or electrons.

In some embodiments, the gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

A microorganism of the present disclosure may classified based on functional characteristics. For example, the microorganism may be or may be derived from a C1-fixing microorganism, an acrobe, a hydrogen-oxidizing bacteria, a hydrogenotroph, an anacrobe, an acetogen, an ethanologen, and/or a carboxydotroph.

### III. CUPRIAVIDUS NECATOR

Cupriavidus necator (also referred to as Hydrogenomonas eutrophus, Alcaligenes eutropha, Ralstonia eutropha, and Wautersia eutropha) is a Gram-negative, flagellated soil bacterium of the Betaproteobacteria class. It is a hydrogen-oxidizing bacterium that is capable of growing in both anaerobic and aerobic environments.

In addition to wild-type and naturally occurring strains of C. necator, the present disclosure also provides non-naturally occurring C. necator strains that are capable of growing in autotrophic conditions at elevated temperatures (i.e., temperature above 30° C.). Such temperature-resistant strains of C. necator are capable of growth even up to 40° C. Naturally occurring strains of C. necator are incapable of growth and survival at temperatures above 30° C. Thus, for the purposes of the present disclosure, the C. necator strain that is engineered to produce unsaturated C18 or unsaturated C16 fatty acids can be a strain that can go in autotrophic conditions between 31-40° C. In other words, the engineered C. nectar of the present disclosure can be prepared from a strain that can grow autotrophically at 31° C., 32° C., 33° C., 34° C., 35° C., 36° C., 37° C., 38° C., 39° C., or at 40° C. The C. nectar may grow at a range of 32-40° C., 33-40° C., 34-40° C., 35-40° C., 31-39° C., 32-39° C., 33-39° C., 34-39° C., or 35-39° C.

The disclosed temperature resistant strains of C. necator were prepared without genetic modification using Adaptive Laboratory Evolution (ALE), as detailed further in Example 1. Briefly, the bacteria is continuously cultivated under clearly defined conditions for prolonged periods of time. The strain deposited at DSM 34774 possesses the desired phenotype characterized by increased temperature tolerance and surprising growth in autotrophic conditions obtained through ALE. However, such temperature resistant strains could feasibly be prepared through genetic modification as well. SEQ ID NOs: 1-3 provide the genome of an ALE-derived strain of C. necator that is temperature resistant and able to grow in autotrophic conditions at up to 40° C., and those skilled in the art could use this information to prepare further temperature resistant strains via genetic modification of a wild-type strain of C. necator. Such temperature resistant C. necator may be used as a chassis or host cell for the introduction of heterologous genes from other species.

C. necator strains of present disclosure, including naturally occurring strains, temperature resistant strains, and other modified strains (e.g., strains modified to minimize the H₂:CO₂ uptake ratio while maximizing intracellular O₂ during autotrophic growth), provide several process advantages for the production of single cell protein for food and feed applications as well as other products. The strains are high in protein content with a favorable amino acid profile, and the disclosed strains can be integrated various processing systems, including processing systems that require increased heat integration schemes and/or limited processing reagents, with relative case. Further, the C. necator bacteria provided herein may be advantageously capable of culture and growth in elevated temperature conditions that may be required for enzyme function, biosynthesis, or any metabolic process that requires elevated temperatures exceeding the tolerance of naturally occurring C. necator bacteria strains.

For the purposes of the present disclosure, the C. necator strain utilized for producing unsaturated C16 and/or C18 fatty acids may be a wild-type or naturally occurring stain that is engineered to express one or more heterologous elongase(s), one or more heterologous desaturase(s), or a combination of one or more heterologous elongase(s) and one or more heterologous desaturase(s). Alternatively, the C. necator strain utilized for producing unsaturated C16 and/or C18 fatty acids may optionally be a non-naturally occurring strain, such as a heat adapted or temperature resistant strain of C. necator that can grow at temperatures above 30° C. As explained herein, such heat adapted or temperature resistant strains may be obtained by genetic modification or by ALE. Additionally or alternatively, the C. necator strain utilized for producing unsaturated C16 and/or C18 fatty acids may optionally be modified—either via ALE or genetic modification—to minimize the H₂:CO₂ uptake ratio while maximizing intracellular O₂ during autotrophic growth.

For the purposes of the present disclosure, the C. necator strain utilized for producing unsaturated C16 and/or C18 fatty acids may have a genome comprising or consisting of the nucleic acid sequence of SEQ ID NOs: 1-3 or a genome comprising or consisting of a nucleic acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NOs: 1-3. Additionally or alternatively, the C. necator strain utilized for producing unsaturated C16 and/or C18 fatty acids may have a genome comprising or consisting of a nucleic acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similarity to SEQ ID NOs: 1-3. The C. necator strain utilized for producing unsaturated C16 and/or C18 fatty acids may also be the strain deposited under accession number DSM 34774, or a strain sharing substantial phenotypic characteristics (e.g., temperature resistance) or genotypic characteristics (e.g., a genome with at least 80% sequence identity to SEQ ID NOs: 1-3 or a genome with at least 80% sequence similarity to SEQ ID NOs: 1-3) with a strain deposited under accession number DSM 34774.

The C. necator strain having identification reference number DSM 34774 was deposited under the provisions of the Budapest Treaty at the German Collection of Microorganisms (DSM) located at Inhoffenstraβe 7B, 38124 Braunschweig, Science Campus Braunschweig-Süd, Germany on Oct. 6, 2023. All restrictions on the availability to the public of the deposited material will be irrevocably removed upon the granting of a patent from the above-identified application. The deposited cultures will be replaced should they die or be destroyed during the enforceable life of any patent issued out of this patent application, for five years after the last request for a sample of the deposited microorganism or for a term of at least thirty (30) years. Samples will be stored under agreements that would make them available beyond the enforceable life of the patent for which the deposit was made.

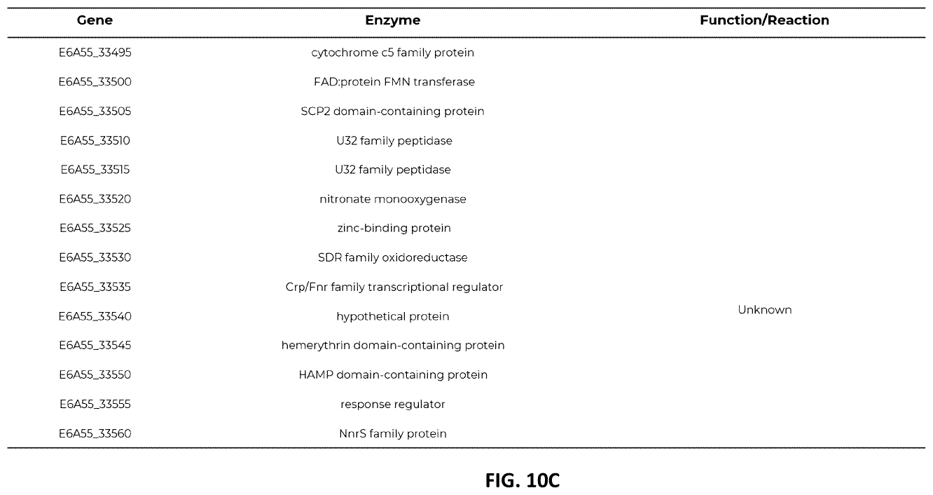

Without being bound by theory, specific examples of genetic modifications that may provide temperature resistance in the non-naturally occurring C1-fixing strain include, but are not limited to, mutations in a hydrogenase regulator (L405H (cTt→cAt) mutation in HoxA encoded by hoxA, E6A55_32285), deletions or frame shifts within certain regions (Δ35433 bp encompassing genes [E6A55_33395]-[E6A55_33560], the open reading frames of which are shown in FIGS. 10A-10C), or mutations in other genes associated with autotrophic growth. Thus, in some embodiments, a temperature resistant strain of C. necator may be genetically modified to possess a temperature resistant phenotype (as opposed to ALE).

Phenotypic features that the C. necator strain utilized for producing unsaturated C16 and/or C18 fatty acids may possess include, but are not limited to, a growth rate that is at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at temperatures above 30° C. For example the growth rate may be at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C. Optionally, the improved growth at high temperatures may be observed when the C. necator is cultured in autotrophic conditions.

In some embodiments, the strain is Cupriavidus necator DSM 428, DSM 531, or DSM541, or any derivatives thereof. In another embodiment, the strain is Cupriavidus necator DSM 34774. In some embodiments, the strain is SEQ ID. NO: 1. In some embodiments, the strain is a derivative of Cupriavidus necator DSM 34774. In another embodiment, the strain is a derivative of SEQ ID. NO: 1.

Additionally or alternatively, the strain of C. necator utilized for the present disclosure may be modified to possess one or more other desirable traits. For example, the strain of C. necator may be modified either via genetic modification or ALE to minimize the H₂:CO₂ uptake ratio while maximizing intracellular O₂ during autotrophic growth. This can be done through the removal or reduction in a membrane-bound hydrogenase system. This can aid in reducing the cost of hydrogen associated with commercial-scale gas fermentation.

A strain of C. necator can be modified to minimize the H₂:CO₂ uptake ratio by deleting all or a portion of, or otherwise reducing the expression of a membrane-bound hydrogenase system (e.g., the hoxKGZ locus). The hoxKGZ locus encodes a multi-subunit membrane-bound hydrogenase system in Cupriavidus necator, and deleting this locus does not inhibit C. necator autotrophic growth on a gas mixture composed of hydrogen, oxygen, and carbon dioxide. Without being bound by theory, this may result because while the soluble hydrogenase system is required for autotrophic growth, the membrane-bound hydrogenase system wastefully oxidizes hydrogen gas, increasing the culture's H₂:CO₂ uptake ratio without benefiting growth. By deleting or reducing expression of the hoxKGZ locus, growth is not only permitted autotrophically, but the H₂:CO₂ uptake ratio is also reduced while increasing the intracellular O₂ availability to improve O₂-dependent product synthesis.

Phenotypic features that the C. necator strain utilized for producing unsaturated C16 and/or C18 fatty acids may possess include, but are not limited to, a H₂:CO₂ uptake ratio that is at least 5%, at least 10%, at least 15%, at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% lower than the H₂:CO₂ uptake ratio of a wild type or naturally occurring strain of C. necator cultured under corresponding temperatures and conditions. For example the H₂:CO₂ uptake ratio may be about 5%, about 10%, about 15%, about 20%, about 25%, about 30%, about 35%, about 40%, about 45%, about 50%, about 55%, as about 60%, about 65%, about 70%, about 75%, about 80%, about 85%, about 90%, about 95%, or about 100% lower than the H₂:CO₂ uptake ratio of a wild type or naturally occurring strain of C. necator cultured under corresponding temperatures and conditions. Optionally, the lower H₂:CO₂ uptake ratio may be observed at elevated culture temperatures, such as 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C. Optionally, the improved growth at elevated culture temperatures may be observed when the C. necator is cultured in autotrophic conditions.

For the purposes of the present disclosure, a strain of C. necator utilized to produce C16 or C18 fatty acids may possess both temperature resistance and a reduced H₂:CO₂ uptake ratio relative to a wildtype strain of C. necator. For example, the strain deposited under accession number DSM 34774, or a strain sharing substantial phenotypic characteristics (e.g., temperature resistance) or genotypic characteristics (e.g., a genome with at least 80% sequence identity to SEQ ID NOs: 1-3 or a genome with at least 80% sequence similarity to SEQ ID NOs: 1-3) with a strain deposited under accession number DSM 34774 may be further modified by deleting all or a portion of, or otherwise reducing the expression of a membrane-bound hydrogenase system (e.g., the hoxKGZ locus), thus providing a strain of C. necator with both desirable features.

### IV. ADAPTIVE LABORATORY EVOLUTION (ALE)

Bacterial strains according to the disclosure can be evolved using adaptive laboratory evolution methods to improve heat tolerance and improve growth and biosynthesis in autotrophic conditions. However, it is understood by those skilled in the art and for the purposes of the present disclosure that ALE is not the only methodology that can be used to prepare a temperature resistant strain of C. necator, but merely one example of a suitable method.

ALE refers to the culture of cells or organisms under defined conditions leading to adaptive changes that accumulate in populations of cells or (microbial) organisms during selection under specified growth conditions. In particular, the desired trait is selected in an evolution environment where it provides a fitness benefit. In the target environment, the desired trait is exploited (for instance, heat resistance at elevated temperature conditions). In the target environment, the desired trait allows the increase or decrease of at least one desired trait and/or avoids the increase or decrease of at least one trait. In one specific embodiment, in the target environment the desired trait allows the increase in the production flux of a least one desired product or survival advantage.

Typically, a metabolic trait or a phenotypic trait of interest evolves over several generations of the cell or organism. This may include at least two generations, e.g., at least 10, at least 50, at least 100, at least 200, at least 300 or more generations, preferably at least 50 generations, more preferably about 100 generations or more of the cell or organism are necessary to evolve a metabolic trait. Thus, the cells or organisms are cultured for a certain time in a desired environment for, such as several days, weeks, months or years.

The target environment should be suitable to evolve a desired trait. When the population of cells or organisms is cultured in the target environment, a part of the population of the cell or organism will establish the desired trait, while some of the cells or organisms will die. In this way, the desired trait is selected. Preferably, at least 5%, at least 10%, at least 20%, at least 30%, at least 40%, at least 50%, at least 60%, at least 70%, at least 80%, at least 90% or more of the population have established the desired metabolic or phenotypic trait.

For the purposes of the present application, continuous ALE can be used to obtain a heat-resistant strain of C. necator capable of growth and culture at elevated temperatures, optionally in autotrophic conditions. As one example of continuous ALE, Example 1 of this disclosure describes inoculating a continuous-flow stirred tank reactor (CSTR) using a base strain of C. necator (H16 PHB-4). The culture can be continuously grown on a minimal autotrophic media with H₂/O₂/CO₂ inputs. Once the culture is established and growth was maintained at a steady state, the reactor temperature was increased step-wise and held to allow the culture to adapt to the increased temperature. These step wise changes may be continued over the course of several days; for example, 1-3 days, 3-50 days, 50-100 days, 50 days to 1 year, or 1-5 years.

The pH can be kept between pH 4 and 12, preferably between pH 6 and 9, particularly preferably between pH 7 and 8.

For the purposes of the present disclosure, ALE can be operated batchwise, semi-batchwise or continuously.

### V. MICROBIAL CULTURES

For the purposes of the present disclosure, C. necator may be grown under any suitable conditions, in an environment that is suitable for growth and production of biomass. For example, the C. necator may be grown in autotrophic culture conditions, heterotrophic culture conditions, or a combination of autotrophic and heterotrophic culture conditions.

A heterotrophic culture may include a suitable source of carbon and energy, such as one or more sugar (e.g., glucose, fructose, sucrose, etc.). An autotrophic culture may include C1 chemicals such as carbon monoxide, carbon dioxide, methane, methanol, formate, and/or formic acid, and/or mixtures containing C1 chemicals, including, but not limited to, various syngas compositions or various producer gas compositions, e.g., generated from low value sources of carbon and energy, such as, but not limited to, lignocellulosic energy crops, crop residues, bagasse, saw dust, forestry residue, or food, through the gasification, partial oxidation, pyrolysis, or steam reforming of said low value carbon sources, that can be used by an oxyhydrogen microorganism or hydrogen-oxidizing microorganism or carbon monoxide oxidizing microorganism as a carbon source and an energy source.

The culture systems used to grow C. necator described herein can be housed in culture vessels known and used in the art. In some embodiments, large scale production in a bioreactor vessel can be used to produce large quantities of a desired molecule and/or biomass. The bioreactor may include a fermentation tank for culturing the microorganisms under specific conditions that promote bacterial culture and growth.

Bioreactor vessels may be used to contain, isolate, and/or protect the culture environment. The culture vessels include those that are known to those of ordinary skill in the art of large scale microbial culturing. Such culture vessels include but are not limited to one or more of the following: airlift reactors; biological scrubber columns; bubble columns; stirred tank reactors; continuous stirred tank reactors; counter-current, upflow, expanded-bed reactors; digesters and in particular digester systems, for example, such known in the art of bioremediation; filters including but not limited to trickling filters, rotating biological contactor filters, rotating discs, soil filters; fluidized bed reactors; gas lift fermenters; immobilized cell reactors; loop reactors; membrane biofilm reactors; pachuca tanks; packed-bed reactors; plug-flow reactors; static mixers; trickle bed reactors; and/or vertical shaft bioreactors.

Microbial culturing may include the commercial production of biomass and/or organic compounds, e.g., protein product as described herein, specifically single cell protein, cell lysate, protein extract, protein-containing extract, protein concentrate, protein isolate, protein hydrolysate, free amino acids, peptides, oligopeptides, fatty acids or combinations thereof, and/or other nutrients, such as, but not limited to vitamins may be performed in bioreactors at large scale (e.g., 500 L, 1,000 L 5,000 L, 10,000 L, 50,000 L, 100,000 L, 1,000,000 L bioreactor volumes and higher).

The C. necator may be grown in a liquid media inside a bioreactor using methods described herein. The bioreactor containing the microorganisms may be constructed of opaque materials that keep the culture in near or total darkness. Bioreactors constructed out of opaque materials such as steel and/or other metallic alloys and/or reinforced concrete and/or fiberglass and/or various high strength plastic materials can be designed to have large working volumes. In some aspects, fermenters constructed of steel or other metallic alloys that are 50,000 liters and greater in volume may be utilized. In some aspects, bioreactors capable of containing positive headspace pressures above ambient pressure may be utilized. In some aspects, the bioreactor comprising the microorganism does not allow light to penetrate part or most or all of its contained liquid volume. The C. necator may be cultured without significant or any exposure to light. In certain such embodiments, net CO₂ consumption still occurs in the absence of light due to chemoautotrophic metabolism and conditions.

In some embodiments, the C. necator are grown and maintained in a medium containing a gaseous carbon source, such as but not limited to syngas, producer gas, or gas mixtures containing H₂ and CO₂, in the absence of light; where such growth is known as chemoautotrophic growth.

In some aspects, food grade CO₂ and/or air that goes through a direct air capture unit may be utilized by the microorganisms for chemoautotrophic growth. CO₂ may be provided from an industrial source, and optionally may be concentrated via a gas separation procedure, thereby resulting in high concentration food grade CO₂.

The bioreactor or fermenter may be used to culture cells through the various phases of their physiological cycle. A bioreactor is utilized for the cultivation of cells, which may be maintained at particular phases in their growth curve. The use of bioreactors is advantageous in many ways for cultivating chemoautotrophic growth. For certain embodiments, protein-rich cell mass, which is used to produce proteins or protein hydrolysates, is grown to high densities in liquid suspension. Generally, the control of growth conditions, including control of dissolved carbon dioxide, oxygen, and other gases such as hydrogen, as well as other dissolved nutrients, trace elements, temperature and pH, is facilitated in a bioreactor. For certain embodiments, protein-rich cell mass, which is used to produce amino acids, peptides, proteins, fatty acids, hydrolysates, extracts, or whole cell products, is grown to high densities and/or grown at high productivities, in liquid suspension within a bioreactor.

Nutrient media, as well as gases, can be added to the bioreactor as either a batch addition, or periodically, or in response to a detected depletion or programmed set point, or continuously over the period the culture is grown and/or maintained. The bioreactor at inoculation may be filled with a starting batch of nutrient media and/or one or more gases at the beginning of growth, and no additional nutrient media and/or one or more gases are added after inoculation. Nutrient media and/or one or more gases may be added periodically after inoculation. For certain embodiments, nutrient media and/or one or more gases are added after inoculation in response to a detected depletion of nutrient and/or gas. Nutrient media and/or one or more gases may be added continuously after inoculation. In some aspects, the added nutrient media does not contain any organic compounds.

In batch culture systems, the conditions (e.g., nutrient concentration, pH, etc.) under which the microorganism is cultivated generally change continuously throughout the period of growth. To avoid the fluctuating conditions inherent in batch cultures, and to improve the overall productivity of the culture system, the microorganisms that are used for the production of protein and/or fatty acids and/or other nutrients may be grown in a continuous culture system, such as a chemostat or a continuous-flow stirred tank reactor (CSTR). In such systems, the culture may be maintained in a perpetual exponential phase of growth by feeding it with fresh medium at a constant rate while at the same time maintaining the volume of the culture constant. A continuous culture system ensures that cells are cultivated under environmental conditions that remain roughly constant. Cells may be maintained in a perpetual exponential phase through the use of a chemostat system. In certain cases, the culture may be maintained in a steady state with a roughly fixed amount of standing biomass maintained in the bioreactor over time. The growth rate of a microorganism in continuous culture may be changed by altering the dilution rate. The growth rate of the microorganism may be changed by altering the dilution rate. In aspects, the continuous bioreactor may be maintained as a turbidostat, where a fixed amount of standing biomass is maintained in the bioreactor over time, and where all surplus biomass that is produced beyond that necessary to maintain the fixed amount of standing biomass within the bioreactor, is harvested continuously from the bioreactor.

Inoculation of the culture into the bioreactor may be performed by methods including, but not limited to, transfer of a C. necator culture from an existing culture inhabiting another bioreactor, or incubation from a seed stock raised in an incubator. The stock of the strain may be transported and stored in forms including but not limited to a powder, liquid, frozen, or freeze-dried form as well as any other suitable form, which may be readily recognized by one skilled in the art. Reserve bacterial cultures may be kept in a metabolically inactive, freeze-dried state until required for restart. When establishing a culture in a very large reactor, cultures may be grown and established in progressively larger intermediate scale vessels prior to inoculation of the full-scale vessel.

Bioreactors may have mechanisms to enable mixing of the nutrient media that include, but are not limited to, one or more of the following: spinning stir bars, blades, impellers, or turbines; spinning, rocking, or turning vessels; gas lifts, sparging; recirculation of broth from the bottom of the container to the top via a recirculation conduit, flowing the broth through a loop and/or static mixers. The culture media may be mixed continuously or intermittently.

The microorganism-containing nutrient medium may be removed from the bioreactor partially or completely, periodically or continuously. The microorganism-containing nutrient medium may be replaced periodically with fresh cell-free medium to maintain the cell culture in an exponential growth phase, and/or in another targeted growth phase (e.g. arithmetic growth), and/or to replenish the depleted nutrients in the growth medium, and/or remove inhibitory waste products.

The ports that are standard in bioreactors may be utilized to deliver, or withdraw, gases, liquids, solids, and/or slurries, into and/or from the bioreactor vessel enclosing the microbes. Many bioreactors have multiple ports for different purposes (e.g., ports for media addition, gas addition, probes for pH and dissolved oxygen, and sampling), and a given port may be used for various purposes during the course of a culture process. As an example, a port might be used to add nutrient media to the bioreactor at one point in time, and at another time might be used for sampling. Preferably, the multiple uses of a sampling port can be performed without introducing contamination or invasive species into the growth environment. A valve or other actuator enabling control of the sample flow or continuous sampling can be provided to a sampling port. The bioreactors may be equipped with at least one port suitable for culture inoculation that can additionally serve other uses including the addition of media or gas. Bioreactor ports enable control of the gas composition and flow rate into the culture environment. For example, the ports can be used as gas inlets into the bioreactor through which gases are pumped.

Gases that may be pumped into a bioreactor include, but not are not limited to, one or more of the following: syngas, producer gas, hydrogen gas, CO, CO₂, O₂, air, air/CO₂ mixtures, natural gas, methane, ammonia, nitrogen, noble gases, such as argon, as well as other gases. In some embodiments the CO₂ pumped into the system may come from sources including, but not limited to: CO₂ from the gasification of organic matter; CO₂ from the calcination of limestone, CaCO₃, to produce quicklime, CaO; CO₂ from methane steam reforming, such as the CO₂ byproduct from ammonia, methanol, or hydrogen production; CO₂ from combustion, incineration, or flaring; CO₂ byproduct of anaerobic or aerobic fermentation of sugar; CO₂ byproduct of a methanotrophic bioprocess; geologically or geothermally produced or emitted CO₂; CO₂ removed from acid gas or natural gas.

One or more gases in addition to carbon dioxide, or in place of carbon dioxide as an alternative carbon source, may either be dissolved into solution and fed to the culture broth and/or dissolved directly into the culture broth, including but not limited to gaseous electron donors and/or carbon sources (e.g., hydrogen and/or CO and/or methane gas). Input gases may include other electron donors and/or electron acceptors and/or carbon sources and/or mineral nutrients such as, but not limited to, other gas constituents and impurities of syngas (e.g., hydrocarbons); ammonia; hydrogen sulfide; and/or other sour gases; and/or O₂; and/or mineral containing particulates and ash.

One or more gases may be dissolved into the culture broth, including but not limited to gaseous electron donors such as, but not limited to, one or more of the following: hydrogen, carbon monoxide, methane, hydrogen sulfide or other sour gases; gaseous carbon sources such as, but not limited to one or more of the following: CO₂, CO, CH₄; and electron acceptors such as, but not limited to, oxygen, either within air (e.g., 20.9% oxygen) or as pure O₂ or as an O₂-enriched gas. The dissolution of these and other gases into solution may be achieved using a system of compressors, flowmeters, and flow valves known to one skilled in the art of fermentation engineering, that feed into one of more of the following widely used systems for dispersing gas into solution: sparging equipment; diffusers including but not limited to dome, tubular, disc, or doughnut geometries; coarse or fine bubble aerators; venturi equipment. Surface aeration and/or gas mass transfer may also be performed using paddle aerators and the like. In certain aspects, gas dissolution may be enhanced by mechanical mixing with an impeller or turbine, as well as hydraulic shear devices to reduce bubble size. Following passage through the reactor system holding microorganisms which uptake the gases, in certain embodiments the residual gases may either be recirculated back to the bioreactor, or burned for process heat, or flared, or injected underground, or released into the atmosphere.

In some aspects, the C. necator may grow on H₂ and CO₂ and other dissolved nutrients under microaerobic conditions. In some aspects, a C1 chemical such as but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, are biochemically converted into longer chain organic chemicals (i.e., C2 or longer and, in some embodiments, C5 or longer carbon chain molecules) under one or more of the following conditions: aerobic, microaerobic, anoxic, anaerobic, and/or facultative conditions.

A controlled amount of oxygen can also be maintained in the culture broth of some. Oxygen may be actively dissolved into solution fed to the culture broth and/or directly dissolved into the culture broth. In some aspects, conditions suitable for growth of an oxyhydrogen microorganism may be deployed, such as use of H₂ and O₂ gas substrates (electron donors and acceptors), and optionally a C1 gaseous carbon source, such as CO₂ and/or CO.

The C. necator may convert a fuel gas, including but not limited to syngas, producer gas, CO, CO₂, H₂, natural gas, methane, and mixtures thereof. In some embodiments, the heat content of the fuel gas is at least 100 BTU per standard cubic foot (scf). In some embodiments, a bioreactor that is used to contain and grow the microorganisms is equipped with fine-bubble diffusers and/or high-shear impellers for gas delivery.

Introducing and/or raising the gas flow rate into a bioreactor can enhance mixing of the culture and produce turbulence if the gas inlet is positioned beneath the surface of the liquid media such that gas bubbles or sparges up through the media. Mixing may be enhanced through turbulence provided by gas bubbles and/or sparging and/or gas plugging up through the liquid media. The bioreactor may include gas outlet ports for gas escape and pressure release. Gas inlets and outlets may be equipped with check valves to prevent gas backflow.

A nutrient media for culture growth and production may be used, including an aqueous solution containing suitable minerals, salts, vitamins, cofactors, buffers, and other components needed for microbial growth, known to those skilled in the art [Bailey and Ollis, Biochemical Engineering Fundamentals, 2nd ed; pp 383-384 and 620-622; McGraw-Hill: New York (1986)].

Chemicals used for maintenance and growth of microbial cultures as known in the art are included in the nutrient media. These chemicals may include but are not limited to one or more of the following: nitrogen sources such as ammonia, ammonium (e.g., ammonium chloride (NH₄Cl), ammonium sulfate ((NH₄)₂SO₄)), nitrate (e.g., potassium nitrate (KNO₃)), urea or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), dipotassium phosphate (K₂HPO₄)); sulfate; yeast extract; chelated iron; potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulfate (MgSO₄ 7H₂O) or magnesium chloride (MgCl₂), calcium chloride (CaCl₂)) or calcium carbonate (CaCO₃), manganese sulfate (MnSO₄7H₂O) or manganese chloride (MnCl₂), ferric chloride (FcCl₃), ferrous sulfate (FeSO₄7H₂O) or ferrous chloride (FeCl₂ 4H₂O), sodium bicarbonate (NaHCO₃) or sodium carbonate (Na₂CO₃), zinc sulfate (ZnSO₄) or zinc chloride (ZnCl₂), ammonium molybdate (NH₄MoO₄) or sodium molybdate (Na₂MoO₄ 2H₂O), cuprous sulfate (CuSO₄) or copper chloride (CuCl₂ 2H₂O), cobalt chloride (CoCl₂ 6H₂O), aluminum chloride (AlCl₃·6H₂O), lithium chloride (LiCI), boric acid (H₃BO₃), nickel chloride NiCl₂ 6H₂O), tin chloride (SnCl₂ H₂O), barium chloride (BaCl₂ 2H₂O), copper selenate (CuScO₄ 5H₂O) or sodium selenite (Na₂SeO₃), sodium metavanadate (NaVO₃), chromium salts).

C. necator strains described herein can be cultured in media of any type (rich or minimal), including fermentation medium, and any composition. The selected medium can be supplemented with various additional components. Some non-limiting examples of supplemental components include glucose, fructose, sucrose, starches, polysaccharides, protein hydrolysates, antibiotics, IPTG for gene induction, and ATCC Trace Mineral Supplement. Similarly, other aspects of the medium and growth conditions described herein may be optimized through routine experimentation. For example, pH and temperature are non-limiting examples of factors which can be optimized. In some embodiments, factors such as choice of media, media supplements, and temperature can influence production levels of a desired molecule. In some embodiments, the concentration and amount of a supplemental component may be optimized. In some embodiments, how often the media is supplemented with one or more supplemental components, and the amount of time that the media is cultured before harvesting the desired molecule is optimized.

The concentrations of nutrient chemicals (e.g., carbon sources, and/or various mineral nutrients), may be maintained within the bioreactor close to or at their respective optimal levels for optimal carbon uptake and/or fixation and/or conversion and/or production of biomass and/or organic compounds, and in particular protein, which may be routinely determined and/or optimized by one of ordinary skill in the art of culturing microorganisms.

One or more of the following parameters may be monitored and/or controlled in the bioreactor: waste product levels; pH; temperature; salinity; dissolved oxygen; dissolved carbon dioxide gas; liquid flow rates; agitation rate; gas pressure. In certain embodiments, the operating parameters affecting chemoautotrophic growth, and/or other types of growth (e.g., heterotrophic growth) are monitored with sensors (e.g., dissolved oxygen probe or oxidation-reduction probe to gauge electron donor/acceptor concentrations), and/or are controlled either manually or automatically based upon feedback from sensors through the use of equipment including but not limited to actuating valves, pumps, and agitators. The temperature of the incoming broth as well as of incoming gases may be regulated by systems such as, but not limited to, coolers, heaters, and/or heat exchangers.

The C. necator culture and bioreaction may be maintained using continuous influx and removal of nutrient medium and/or biomass, in steady state where the cell population and environmental parameters (e.g., cell density, pH, DO, chemical concentrations) are targeted at a constant level over time.

In some aspects, the pH of the microbial culture is controlled. pH may be controlled within an optimal range for microbial maintenance and/or growth and/or conversion of feedstock and/or production of organic compounds and/or survival. To address a decrease in pH, a neutralization step can be performed directly in the bioreactor environment or prior to recycling the media back into the culture vessel through a recirculation loop. Neutralization of acid in the broth of certain embodiments can be accomplished by the addition of bases, including but not limited to one or more of the following: limestone, lime, sodium hydroxide, ammonia, ammonium hydroxide, caustic potash, magnesium oxide, iron oxide, alkaline ash.

The culture systems may be carried out at varying temperatures controlled by the bioreactor system. The temperature of the culture system may be controlled to optimize enzyme function, protein and/or fatty acid biosynthesis, or biomass production. Additionally or alternatively, the temperature of the culture system may be dictated by the incorporation of the culture system into an industrial process with a temperature above 30° C. In such situations, the temperature of the culture system may be 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C.

### VI. ENGINEERED STRAINS OF C. NECATOR FOR PRODUCING UNSATURATED FATTY ACIDS

Prior to the present application, the precise fatty acid profile of C. necator was not known, and the present disclosure shows, for the first time, that C. necator is intrinsically low in nutritionally valuable polyunsaturated fatty acids, particularly C18 polyunsaturated fatty acids. Further, Applicant has discovered that C. necator the overall amount of long chain saturated fatty acids (e.g.: C16:0 and C18:0) produced by C. necator is relatively low, and that unsaturated fatty acid synthesis can be further improved with the expression of additional elongases or other enzymes involved in fatty acid biosynthesis. As provided in more detail below, the engineered strains of C. necator provided herein are capable of producing monounsaturated and polyunsaturated fatty acids.

The engineered C. necator strains of the present disclosure express at least one heterologous desaturase enzyme, which may be encoded by a transgene. By expressing the heterologous desaturase enzyme(s), the strain of C. necator can synthesize at least one unsaturated C18 fatty acid that is not natively produced by C. necator. The unsaturated C18 fatty acid may be selected from an oleic acid, a linoleic acid, an α-linoleic acid, a γ-linolenic acid, a linolelaidic acid, an elaidic acid, a vaccenic acid, a stearidonic acid, and any combination thereof.

The engineered strains of C. necator may also be able to synthesize an increased amount of an unsaturated fatty acid relative to a strain of C. necator that does not express the heterologous desaturase enzyme(s). The unsaturated fatty acid may be a C16 fatty acid, a C18 fatty acid, or both a C16 fatty acid and a C18 fatty acid.

The unsaturated fatty acids produced by the disclosed engineered strains and processes are generally C16 or C18 fatty acid molecules. The unsaturated C16 or C18 fatty acid molecules may have at least two double bonds or at least three, four, five, or six double bonds. These C16 or C18 fatty acid molecules can be isolated from the organism in the form of an oil or as free fatty acid. The C16 fatty acids may include, but are not limited to, a palmitoleic acid, a sapienic acid, and any combination thereof. The C18 fatty acids may include, but are not limited to, an oleic acid, a linoleic acid, an α-linoleic acid, a γ-linolenic acid, a linolelaidic acid, an elaidic acid, a vaccenic acid, a stearidonic acid, and any combination thereof.

### A. Heterologous Desaturases

Transgenes encoding various desaturase enzymes that may be incorporated into and/or express by the engineered strains of C. necator include, but are not limited to, transgenes that encode a Δ9-desaturase, a Δ12-desaturase, a Δ15-desaturase, a Δ6-desaturase, a Δ5-desaturase, and a Δ4-desaturase. In other words, the heterologous desaturase enzymes that are expressed by the engineered strains of C. necator include, but are not limited to, one or more Δ9-desaturase, Δ12-desaturase, Δ15-desaturase, Δ6-desaturase, Δ5-desaturase, or Δ4-desaturase or any combination thereof. Hence, for the purposes of the present disclosure, various heterologous desaturases can be combined in a particular way to engineer a biosynthetic route to obtain desirable ω6 and ω3 fatty acids.

The nucleic acid sequences that encode proteins with Δ9-desaturase, Δ12-desaturase, Δ15-desaturase, Δ6-desaturase, Δ5-desaturase, and Δ4-desaturase activity can be codon optimized for expression in C. necator regardless of the native species from which they are derived. The nucleic acids can be transfected into the disclosed engineered C. necator alone or in combination, for example, in an expression cassette (i.e., a nucleic acid construct) that the allows expression of the nucleic acids in the C. necator. There can be more than one nucleic acid sequence encoding a protein with a desired enzymatic activity in the nucleic acid construct, such as, for example, a Δ9-desaturase, a Δ12-desaturase, a Δ15-desaturase, a Δ6-desaturase, a Δ5-desaturase, and a Δ4-desaturase may be included. In some embodiments, a Δ9-desaturase and a Δ12-desaturase may be co-expressed. In some embodiments, a Δ9-desaturase, a Δ12-desaturase, and a Δ15-desaturase may be co-expressed.

The copy number of the heterologous Δ9-desaturase, Δ12-desaturase, Δ15-desaturase, Δ6-desaturase, Δ5-desaturase, and/or Δ4-desaturase genes can be increased, so that the level of protein expression is increased and the yield, production and/or production efficiency of the desired unsaturated fatty acids is also increased. The strategy of increasing copy number of the transgene or expression level of the heterologous protein applies analogously to the combination with further desaturases, elongases, or additional enzymes involved in fatty acid and lipid metabolism or biosynthesis.

Heterologous desaturases that can be expressed in C. necator to increase unsaturated fatty acid synthesis or produce C18 unsaturated fatty acids that are not naturally produced by C. necator include, but are not limited to DesC, DesA, and/or DesB from Synechococcus sp. In particular, an engineered strain of C. necator can express DesC alone or in combination with one or both of DesA or DesB. Additionally, an engineered strain of C. necator can express DesA alone or in combination with one or both of DesC or DesB. Additionally, an engineered strain of C. necator can express DesB alone or in combination with one or both of DesC or DesA.

A DesC from a Synechococcus sp. that can be expressed by an engineered strain of C. necator can comprise or consist of a wildtype DesC amino acid sequence or a derivative thereof, which has at least 50% identity or similarity (e.g., at least 60%, at least 70%, at least 80%, at least 90%, at least 95%, at least 96%, at least 97%, at least 98%, or at least 99%) at the amino acid level and maintains Δ9-desaturase activity.

A DesA from a Synechococcus sp. that can be expressed by an engineered strain of C. necator can comprise or consist of a wildtype DesA amino acid sequence or a derivative thereof, which has at least 50% identity or similarity (e.g., at least 60%, at least 70%, at least 80%, at least 90%, at least 95%, at least 96%, at least 97%, at least 98%, or at least 99%) at the amino acid level and maintains desaturase activity.

A DesB from a Synechococcus sp. that can be expressed by an engineered strain of C. necator can comprise or consist of a wildtype DesB amino acid sequence or a derivative thereof, which has at least 50% identity or similarity (e.g., at least 60%, at least 70%, at least 80%, at least 90%, at least 95%, at least 96%, at least 97%, at least 98%, or at least 99%) at the amino acid level and maintains desaturase activity.

It is, however, to be understood that the engineered strains of C. necator disclosed herein are not limited to expression of desaturases (e.g., DesC, DesA, and/or DesB) from Synechococcus sp., but rather may express any number or combination of suitable exogenous or heterologous desaturases.

Suitable exogenous or heterologous nucleic acid sequences that that encode a protein with Δ9-desaturase activity include, but are not limited to, a nucleic acid sequence with the sequence shown below:

ATGCTGTCACGTGCCTGCGTCTACCACGTCGGCCAGTGGAAGAAGTACG
GTGCCAGCGCCTACCACGTCCCCCAGTACAATTTCCAGAAGGGCTACTT
CATGACGGTCGCGACCTCCCAGAAGTTGCCGCTGGACTGGACGACCATC
ATCTACTTCTCGTTGATCCATCTTGTGGCGCTGCTGGCCTTCCTGCCGG
GCAACTTCAGCTGGCAGGCCGTCGGCGTGTTCCTGCTGTTCCACTGGAT
TACCGGCGGCATCGGCATCACGCTCGGCTTCCACCGGCTGGTGTCGCAC
AGGTCCTTTGAAGTGCCCAAGTGGCTGGAATACTTCCTGATCTTCTGCG
GCACGCTGGCCTGCCAGGGCGGCCCGATCGACTGGATCGGGCTGCACCG
TATCCACCACAAGCACTCGGACAATACGCCCGATCCGCATGATTCCAAC
AAGGGCTTCTGGTGGTCGCATATCGGCTGGATGCTCTTCGAGATCCCCG
CGCGCGGCGACATCGATCGCTATATCAAGGACATCAAGGACGATCCCTT
CTACAACTTCTGCCAGAAGTACATGGTGCTGATCCAGGTTGCGCTGGGC
CTGGCGCTGTACGCCTGGGGCGAGGCCTGGGTCGGCAACGGCTGGTCGT
TTGTCATCTGGGGCGTGTTCCTGCGCCTGGCGGTGGTGTTCCACTGTAC
CTGGTTCGTCAACAGCGCGACCCACAAATTCGGCTACAAGAGCCACGAG
AGCAACGATCATTCCAAGAACTGCTGGTGGGTCGCGCTGGTGACCTACG
GCGAAGGCTGGCACAACAACCACCACGCCTACCAGTACTCCGCACGCCA
CGGCCTGAACTGGTGGGAGATCGACATGACCTGGATGACGATCCGCTTC
CTCCAGGCGCTGGGGCTGGCCAAGAATATCCGCCTGGCACCCGCGAAAT
AA.

Suitable exogenous or heterologous nucleic acid sequences encoding a protein with Δ9-desaturase activity may also include derivatives of the nucleic acid sequence shown above, which include sequences that code for proteins or polypeptides having Δ9-desaturase activity with at least 50% identity (e.g., at least 60%, at least 70%, at least 80%, at least 90%, at least 95%, at least 96%, at least 97%, at least 98%, or at least 99%) at the amino acid level with the following sequence: MLSRACVYHVGQWKKYGASAYHVPQYNFQKGYFMTVATSQKLPLDWTTIIYFSLIH LVALLAFLPGNFSWQAVGVFLLFHWITGGIGITLGFHRLVSHRSFEVPKWLEYFLIFC GTLACQGGPIDWIGLHRIHHKHSDNTPDPHDSNKGFWWSHIGWMLFEIPARGDIDRY IKDIKDDPFYNFCQKYMVLIQVALGLALYAWGEAWVGNGWSFVIWGVFLRLAVVF HCTWFVNSATHKFGYKSHESNDHSKNCWWVALVTYGEGWHNNHHAYQYSARHG LNWWEIDMTWMTIRFLQALGLAKNIRLAPAK. Suitable exogenous or heterologous nucleic acid sequences encoding a protein with Δ9-desaturase activity may also include nucleic acid sequences which can be derived as a result of the degenerate genetic code from the amino acid sequence identified above as well as functional equivalents with Δ9-desaturase activity.

Suitable exogenous or heterologous nucleic acid sequences that that encode a protein with Δ12-desaturase activity include, but are not limited to, a nucleic acid sequence with the sequence shown below:

ATGACATCCGTGACCGTGCGCCCGTCCGCAACAACCCTGCTGGAGAAGC
ACCCCAACCTGCGCCTGCGGGACATCCTGGACACGCTGCCGCGCTCGGT
CTACGAGATCAACCCGCTCAAGGCGTGGTCGCGCGTGCTGCTGTCGGTG
GCCGCGGTGGTGGGCTGCTATGCGCTGCTGGCGATCGCGCCCTGGTACC
TGCTGCTGCCTGTCTGGTTCCTGACCGGCACAACCCTGACCGGCTTCTT
CGTGATCGGCCACGACTGCGGCCATCGCAGCTTCTCGCGCAAGAACTGG
GTGAACAACCTGGTCGGCCATCTGGCCTTCCTGCCGCTGATCTACCCGT
TCCATTCGTGGCGCATCCTGCACAACCACCACCATCGCTATACCAACAA
CATGGACGAGGACAACGCGTGGGCGCCGTTCACGCCGGAGCTGTACGAC
GATTCGCCCGCGTTCATCAAGGCCGTCTATCGCGCCATCCGCGGCAAGC
TGTGGTGGCTGGCGTCGGTCATCCACCAGCTCAAGCTGCACTTCAACTG
GTTCGCCTTCGAGGGCAAACAGCGTGAACAGGTGCGCTTCTCGGCGCTG
TTTGTGATCATCGCAGGCGCCATCGCGTTCCCGGTCATGTTCTACACGC
TGGGCGTGTGGGGCGTGGTGAAGTTCTGGCTGATGCCGTGGCTGGGCTA
CCATTTCTGGATGTCCACGTTCACGCTGGTGCACCATACCGTGCCGGAG
ATCCCGTTCTCTTACCGCGACAAGTGGAACGAGGCCATCGCCCAGCTGA
GCGGGACCGTGCACTGCGACTATCCGAAATGGGTCGAAGTGCTGTGCCA
CGATATCAACGTCCACGTGCCGCACCATCTGTCGACCGGCATCCCCAGC
TACAACCTGCGCAAGGCCTATGCCTCGATCAAACAGAACTGGGGCGAAT
ACCTGTACGAAACCAAGTTCTCCTGGGAACTGATGAAGGCCATCACGGA
ACAGTGCCATCTGTATGACGCCGAGCACAACTACATCAGCTTCGCACAG
CACCAGAAGAGATAA

Suitable exogenous or heterologous nucleic acid sequences encoding a protein with Δ12-desaturase activity may also include derivatives of the nucleic acid sequence shown above, which include sequences that code for proteins or polypeptides having Δ12-desaturase activity with at least 50% identity or similarity (e.g., at least 60%, at least 70%, at least 80%, at least 90%, at least 95%, at least 96%, at least 97%, at least 98%, or at least 99%) at the amino acid level with the following sequence: MTSVTVRPSATTLLEKHPNLRLRDILDTLPRSVYEINPLKAWSRVLLSVAAVVGCYA LLAIAPWYLLLPVWFLTGTTLTGFFVIGHDCGHRSFSRKNWVNNLVGHLAFLPLIYPF HSWRILHNHHHRYTNNMDEDNAWAPFTPELYDDSPAFIKAVYRAIRGKLWWLASVI HQLKLHFNWFAFEGKQREQVRFSALFVIIAGAIAFPVMFYTLGVWGVVKFWLMPWL GYHFWMSTFTLVHHTVPEIPFSYRDKWNEAIAQLSGTVHCDYPKWVEVLCHDINVH VPHHLSTGIPSYNLRKAYASIKQNWGEYLYETKFSWELMKAITEQCHLYDAEHNYIS FAQHQKR. Suitable exogenous or heterologous nucleic acid sequences encoding a protein with Δ12-desaturase activity may also include nucleic acid sequences which can be derived as a result of the degenerate genetic code from the amino acid sequence identified above as well as functional equivalents with Δ12-desaturase activity.

Suitable exogenous or heterologous nucleic acid sequences that that encode a protein with Δ15-desaturase activity include, but are not limited to, a nucleic acid sequence with the sequence shown below:

ATGCAATCGACGGTACGCAGCCCGGGCAGCCGCGAGTCGCTGCGCCAGG
ATCTGCCGTTCACGCTCAAGGACGTCAAGGCAGCGATCCCCGACTACTG
CTTCCAGCCGAGCGTGTTCCGCAGCCTGGCCTACTTCTTCCTGGACATC
GGCATCATCGCCGGGCTCTACGCCATCGCCGCCTACCTCGACAGCTGGT
TCTTCTACCCGATCTTCTGGTTTGCCCAGGGTACCATGTTCTGGGCGCT
GTTCGTGGTCGGCCATGACTGCGGCCACGGCTCGTTCAGCCGCTCGAAG
TTCCTGAACGATCTGATCGGCCATCTGTCGCACACGCCGATCCTGGTGC
CCTTCCACGGCTGGCGCATCAGCCACCGCACGCACCACAGCAATACCGG
CAACATCGATACCGACGAATCGTGGTACCCCATCCCGGAAAGCAAGTAT
GATCAGATGGGCTTTGCCGAGAAGCTGGTGCGCTTCTATGCGCCGCTGA
TCGCCTACCCGATCTACCTGTTCAAGCGCTCGCCGGGGCGTGGCCCGGG
CTCGCACTTCAGCCCGAAGAGCCCGCTGTTCAAGCCCGCCGAGCGCAAC
GATATCATCCTGTCGACCGCGGCCATCATCGCAATGGTCGGCTTCCTGG
GCTGGTTCACGGTCCAGTTCGGACTGCTGGCCTTCGTCAAGTTCTACTT
CGTGCCCTATGTGATCTTCGTCATCTGGCTCGATCTGGTGACCTACCTG
CACCATACCGAGGCCGACATCCCCTGGTACCGCGGCGACGACTGGTACT
ATCTCAAGGGCGCGCTGTCGACCATCGACCGCGACTACGGGATCTCAAC
GAGATCCACCATAACATCGGTACGCACGTCGCGCACCATATCTTCCATA
CCATCCCCCACTACCACCTCAAGGACGCGACCGAGGCGATCAAGCCGCT
GCTGGGCGACTACTACCGCGTCAGCCATGCGCCCATCTGGCGCTCGTTC
TTCCGCAGCCAGAAGGCATGCCACTACATCGCCGATCAGGGCTCGCATC
TGTACTACCAGCCCAAGAAATAA

Suitable exogenous or heterologous nucleic acid sequences encoding a protein with Δ15-desaturase activity may also include derivatives of the nucleic acid sequence shown above, which include sequences that code for proteins or polypeptides having Δ15-desaturase activity with at least 50% identity or similarity (e.g., at least 60%, at least 70%, at least 80%, at least 90%, at least 95%, at least 96%, at least 97%, at least 98%, or at least 99%) at the amino acid level with the following sequence: MQSTVRSPGSRESLRQDLPFTLKDVKAAIPDYCFQPSVFRSLAYFFLDIGIIAGLYAIA AYLDSWFFYPIFWFAQGTMFWALFVVGHDCGHGSFSRSKFLNDLIGHLSHTPILVPF HGWRISHRTHHSNTGNIDTDESWYPIPESKYDQMGFAEKLVRFYAPLIAYPIYLFKRS PGRGPGSHFSPKSPLFKPAERNDIILSTAAIIAMVGFLGWFTVQFGLLAFVKFYFVPYV IFVIWLDLVTYLHHTEADIPWYRGDDWYYLKGALSTIDRDYGIFNEIHHNIGTHVAH HIFHTIPHYHLKDATEAIKPLLGDYYRVSHAPIWRSFFRSQKACHYIADQGSHLYYQP KK. Suitable exogenous or heterologous nucleic acid sequences encoding a protein with Δ15-desaturase activity may also include nucleic acid sequences which can be derived as a result of the degenerate genetic code from the amino acid sequence identified above as well as functional equivalents with Δ15-desaturase activity.

Other suitable exogenous or heterologous desaturases include, but are not limited to, desaturases with Δ6-desaturase activity, Δ5-desaturase activity, and/or Δ4-desaturase activity.

In certain aspects, the C. necator may be engineered to express one or more functional fragments of a desaturase. The phrase “functional fragment” as used herein refers to a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length, polypeptide. The functional fragment can generally, but not always, be comprised of a continuous region of the protein, wherein the region has functioning desaturase activity.

Heterologous proteins involved in fatty acid biosynthesis and the transgenes that express them can be derived from a variety of organisms that natively produce desirable fatty acids, such as certain bacteria, cyanobacteria, fungi, algae, and other microbes or animals.

Thus, heterologous desaturases, as well as nucleic acid sequences encoding the same, can be derived from a cyanobacteria such as a cyanobacteria from the genera Synechococcus. The Synechococcus species can be selected from Synechococcus ambiguus Skuja, Synechococcus arcuatus, Synechococcus bigranulatus, Synechococcus brunneolus, Synechococcus caldarius, Synechococcus capitatus. Synechococcus carcerarius, Synechococcus elongatus, Synechococcus endogloeicus, Synechococcus epigloeicus, Synechococcus ferrunginosus, Synechococcus intermedius, Synechococcus koidzumii, Synechococcus lividus, Synechococcus marinus, Synechococcus minutissimus, Synechococcus mundulus, Synechococcus nidulans, Synechococcus rayssae, Synechococcus rhodobaktron, Synechococcus roseo-persicinus, Synechococcus roseo-purpureus, Synechococcus salinarum, Synechococcus salinus, Synechococcus sciophilus, Synechococcus sigmoideus, Synechococcus spongiarum, Synechococcus subsalsus, Synechococcus sulphuricus, Synechococcus vantieghemii, Synechococcus violaceus, Synechococcus viridissimus, and Synechococcus vulcanus. In some embodiment, the desaturase is derived from Synechococcus elongatus.

Additionally or alternatively, the heterologous desaturases, as well as nucleic acid sequences encoded the same, can be derived from a bacteria such as E. coli, a Sterptomyces sp., a Psuedomonas sp., a Clostridia sp., or a Bacillus sp.

Additionally or alternatively, the heterologous desaturases, as well as nucleic acid sequences encoded the same, can be derived from algae, for example algae from the Prasinophyceae family, such as from the genera Heteromastix, Mammella, Mantoniella, Micromonas, Nephroselmis, Ostreococcus, Prasinocladus, Prasinococcus, Pseudoscourfielda, Pycnocetraselis, and Pycnococas. Particular species include, but are not limited to Heteromastix longifillis, Mamiella gilva, Mantoniella squamata, Micromonas pusilla, Nephroselmis olivacea, Nephroselmis pyriformis, Nephroselmis rotunda, Ostreococcus tauri, Ostreococcus sp. Prasinocladus ascus, Prasinocladus lubricus, Pycnococcus provasolii, Pyramimonas amylifera, Pyramimonas disomata, Pyramimonas obovata, Pyramimonas orientalis, Pyramimonas parkeae, Pyramimonas spinifera, Pyramimonas sp., Tetraselmis apiculata, Tetraselmas Tetraselmis Tisetraselmis Tetraselmis Tisetraselmis Tetraselmis Tisetraselmis Tetraselmis Tis hazeni, Tetraselmis impellucida, Tetraselmis inconspicua, Tetraselmis levis, Tetraselmis maculata, Tetraselmis marina, Tetraselmis striata, Tetraselmis subcordiformis, Tetraselmis suecica, Tetraselmis tetrabrachia, Tetraselmisetrosa rubens, or Tetraselmis sp. Additionally contemplated are algae of the Euglenaceae family such as from the genera Ascoglena, Astasia, Colacium, Cyclidiopsis, Euglena, Euglenopsis, Hyalophacus, Khawkinea, Lepocinclis, Phacus, Strombomonas, or Trachelomonas Particular species include, but are not limited to, Euglena acus, Euglena genicaoclenatais, Euglena geniculatais Euglena rostrifera, Euglena viridis, Colacium stentorium, Trachelomonas cylindrica or Trachelomonas volvocina.

### B. Heterologous Elongases and Additional Enzymes

While C. necator is suited for various commercial applications due to its ability to culture and proliferate in a variety of processing environments and produce a large amount of protein, C. necator exhibits low fatty acid production, in general. More specifically, C. necator does not naturally produce significant amounts of long chain saturated fatty acids, which presents a challenge to the biosynthesis of desirable C16 and C18 unsaturated fatty acids. Accordingly, the present disclosure provides for the introduction of nucleic acids encoding various elongases or other enzymes to increase the production of long-chain saturated fatty acids. Thus, the engineered C. necator of the present disclosure may be modified to express one or more elongases or other enzymes involved in fatty acid biosynthesis in order to increase the overall amount of long-chain saturated fatty acids that are present and that can then be converted to unsaturated fatty acids (e.g., ω-3 and/or ω-6 fatty acids). By improving long chain saturated fatty acid synthesis in C. necator relative to a wild type strain, the resulting engineered bacterium is capable of producing higher levels of the necessary precursor elements for C16 and C18 unsaturated and polyunsaturated fatty acids. Accordingly, various elongase enzymes may be used in combination with the above-disclosed desaturases to provide a further engineered biosynthetic pathway for the production of C16 and C18 unsaturated and polyunsaturated fatty acids (e.g., ω-3 and/or ω-6 fatty acids).

Elongases that may be expressed in the engineered C. necator of the present disclosure include, but are not limited to, 45-elongases, 46-elongases or 49-elongases.

Suitable exogenous or heterologous nucleic acid sequences that that encode a protein with serine protease activity include, but are not limited to, a nucleic acid sequences that code for proteins or polypeptides having serine protease activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Elongation of fatty acids protein (EC 2.3.1.199) (Very-long-chain 3-oxoacyl-CoA synthase) from Diacronema viridis

MAKSAVQQEAERLTAGLWLPMLLSAGYLLVLSANRASFHNNIDHPNGAY
STAWPIVPTAMTAFYLAMVFGHTKYFESRKPMSGLKDYMFTYNLYQVII
NVWCVVAFCVEVRRAGMSVIGNKVDLGPNSFRLGFVTWVHYNNKYVELL
DTLWMVLRKKSSQVSFLHVYHHCLLIWAWFIVIKFGNGGDAYFGGMLNS
LIHVMMYSYYTMALLGWSCPWKRYLTQAQLVQFCICLTHSTWAAVTGVY
PWKICLVEMWVMISMLVLFTRFYNQSYAKEKSAKDAAALLQAKAATAKA
QQTSGNFAPSLRRIAKHEKG

Elongation of fatty acids protein (EC 2.3.1.199) (Very-long-chain 3-oxoacyl-CoA synthase) from Isochrysis galbana (Marine planktonic alga)

MATEATASIWAAVSDPEILIGTFSYLLLKPILRSSGLVDEKKGAYRTSM
IWYNVILALFSATSFYVTATALGWDYGSGEWLRRLTGDTPQPLFQCPSR
VWDSKLFVWTAKAFYYSKYVEYLDTAWLVLKGKNVSFLQAFHHFGAPWD
VYLGIRLQNEGVWIFMFFNSFIHTIMYTYYGLTAAGYKIKAKPLITAMQ
ISQFMGGFILVWDYINIPCFRSDNGKVFSWVFNYAYVGFVFLLFCHFFY
KDNLASKKPAKGGKAL

Polyunsaturated fatty acid specific elongation enzyme 1 from Physcomitrium patens (Spreading-leaved earth moss)

MEVVERFYGELDGKVSQGVNALLGSFGVELTDTPTTKGLPLVDSPTPIV
LGVSVYLTIVIGGLLWIKARDLKPRASEPFLLQALVLVHNLFCFALSLY
MCVGIAYQAITWRYSLWGNAYNPKHKEMAILVYLFYMSKYVEFMDTVIM
ILKRSTRQISFLHVYHHSSISLIWWAIAHHAPGGEAYWSAALNSGVHVL
MYAYYFLAACLRSSPKLKNKYLFWGRYLTQFQMFQFMLNLVQAYYDMKT
NAPYPQWLIKILFYYMISLLFLFGNFYVQKYIKPSDGKQKGAKTE

In certain aspects, the C. necator may be engineered to express one or more functional fragments of an elongase. That is, the functional fragment is a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length, polypeptide. The functional fragment can generally, but not always, be comprised of a continuous region of the protein, wherein the region has functioning elongase activity.

Additional enzymes that may be expressed in the engineered C. necator of the present disclosure include, but are not limited to, FabA, FabB, FabD, FabF, FabH, and FabZ derived from E. coli.

Suitable exogenous or heterologous nucleic acid sequences that that encode a protein with FabF activity include, but are not limited to, a nucleic acid sequence with the sequence shown below:

ATGAGCCGCCGTAGGGTAGTGGTGACCGGCCTCGGCCTCGTATCGCCCG
TCGGCAATACCGTCGCCGAAGGCTGGGCCAACCTGGTGGCGGGCAAGAG
CGGCATCGCCACGATTACCAAGTTCGATCACTCGGCGCTCAGCGTCCAT
TTCGCAGGCGAGGTCAAGGGCTTCAATGCGGAAGACTACATCCCCGCCA
AGGAAGCGCGCGCGATGGATACCTTCATCCATTTCGGCATCGCGGCCGG
AACCCAGGCGCTGAAGGATTCGGGCCTGGAAGTGACCGAGGCCAATGCC
GAGCGCATCGGCGTGCTGGTCGGCTCGGGCATCGGCGGCCTGCCGCTGA
TCGAGGAAACGCATGCGGTGCTGACCGAGCGCGGCCCGCGCCGCATCAG
CCCGTTCTTCGTCCCGGGCTCGATCATCAACATGATTGCGGGCCATCTG
TCGATCATCCACGGCATCAAGGGCCCCAACCTGGCGGCGGTGACCGCCT
GCACGACCGGCCTGCATTCGATCGGCCTGGCCGCGCGCCTGATCCAGGC
AGGCGATGCCGACGCCATGCTGGCGGGCGGCGCTGAATCGACCGTGTCG
CCGCTGGGCATCGGCGGCTTTGCCGCCGCGCGGGCGCTGTCCACGCGCA
ATGACGATCCCGCCGCCGCCTCGCGTCCGTGGGACAAGGATCGCGACGG
CTTCGTGCTCGGCGAGGGTGCGGGCGTGATGATGCTGGAAGAGTACGAG
TCGGCCAAGGCACGCGGGGCGCGCATCTACGCCGAGCTGATCGGCTTCG
GCATGTCCGGCGACGCCTACCACATGACCGCACCCAATATGGACGGCCC
GCGCCGCTGCATGGTCAATGCGCTCAAGGATGCGGGCATCAATACCGAT
CAGGTACACTACCTCAATGCGCACGGAACCAGCACTCCGCTGGGCGACA
AGAACGAGAGCGATGCCATCAAGGCCGCCTTCGGCGATCAGGCCTACAA
GATGGTCGTGAACTCGACCAAGTCGATGACCGGCCATCTGCTGGGCGGC
GCGGGCGGCCTGGAAAGCGTCTTTACCGTGCTGGCCCTGCACAACCAGG
TCAGCCCGCCCACGATCAACCTGGACAACCAGGATCCGGAGTGCGATCT
CGACTACGTCGCCAATACCGCCCGCGAGATGAAGATCGAAGTGGCGGTC
AAGAACAACTTCGGGTTTGGCGGCACCAATGGCACGCTGGTGTTCCGCC
GGGCATAA

Suitable exogenous or heterologous nucleic acid sequences encoding a protein with FabF activity may also include derivatives of the nucleic acid sequence shown above, which include sequences that code for proteins or polypeptides having FabF activity with at least 50% identity or similarity (e.g., at least 60%, at least 70%, at least 80%, at least 90%, at least 95%, at least 96%, at least 97%, at least 98%, or at least 99%) at the amino acid level with the following sequence: MSRRRVVVTGLGLVSPVGNTVAEGWANLVAGKSGIATITKFDHSALSVHFAGEVKG FNAEDYIPAKEARAMDTFIHFGIAAGTQALKDSGLEVTEANAERIGVLVGSGIGGLPL IEETHAVLTERGPRRISPFFVPGSIINMIAGHLSIIHGIKGPNLAAVTACTTGLHSIGLAA RLIQAGDADAMLAGGAESTVSPLGIGGFAAARALSTRNDDPAAASRPWDKDRDGFV LGEGAGVMMLEEYESAKARGARIYAELIGFGMSGDAYHMTAPNMDGPRRCMVNA LKDAGINTDQVHYLNAHGTSTPLGDKNESDAIKAAFGDQAYKMVVNSTKSMTGHL LGGAGGLESVFTVLALHNQVSPPTINLDNQDPECDLDYVANTAREMKIEVAVKNNF GFGGTNGTLVFRRA. Suitable exogenous or heterologous nucleic acid sequences encoding a protein with FabF activity may also include nucleic acid sequences which can be derived as a result of the degenerate genetic code from the amino acid sequence identified above as well as functional equivalents with FabF activity.

Suitable exogenous or heterologous nucleic acid sequences that that encode a protein with FabD activity include, but are not limited to, a nucleic acid sequence with the sequence shown below:

ATGAAGTTTGCATTTGTCTTCCCGGGACAAGGCAGCCAGAGCGTCGGCA
TGCTCAATGCCTTCGCCGACAATGCGGTGGTGCGCGCGACCGTCGAGGA
AGCCTCGGCCGCGCTGGGCCAGGATCTCGGCCGCCTGATTGCCGAAGGC
CCGGCGGAAGAACTGAACCTGACGACCAATACCCAGCCCGTGATGCTGA
CCGCTGCCGTGGCCGTCTACCGTGCCTGGCTGGATGCGGGCGGCCCGGC
ACCGGCGCTGGTGGCGGGCCATTCGCTGGGCGAGTATTCCGCCCTGGTG
GCCGCGGGCGTGATCCCGTTTGCCGATGCGGTGCCGCTGGTGCGCTTCC
GTGCCCAGGCGATGCAAGAGGCCGTTCCGGTAGGCGAGGGTGCGATGGC
CGCCATCCTCGGGTTGAGCGACGACGACGTCCGTGCCGCCTGCGCCGAG
GCCTCGGCCGCCGGTGTTGTCGAAGCGGTCAACTTCAACGCCCCAAGCC
AAGTGGTGATCGCGGGCCACAAGGCCGCGGTCGAGAAGGCCTGCGAAAT
CGCGAAGGGCAAGGGTGCCAAGCGCGCGCTGCCGCTGCCGGTGTCGGCT
CCGTTCCACTCGTCGCTGCTCAAGCCCGCCTCGGATCGGCTGCGCGAGC
GCATGGCGGGCCTGGCCTTCAGCGCCCCGTCGATCCCGCTGGTCAACAA
CGTCGACGTGGCCATCGTCAATGATCCGGATGCCATCAAGGATGCGCTG
GTGCGCCAGGCCGCGGCACCGGTGCGCTGGGTCGAGTGCGTCCAGAAGA
TGGCCGCCGAAGGCGTCACGCACGTGATCGAGTGCGGGCCGGGCAAGGT
GCTGGCTGGCATGACCAAGCGCATCGACGGCAACCTGACCGGCGGCGCG
ATCTTCGATCCCGCCAGCCTCCAGGATACCCTGGCGCTGCTTAAATAA

Suitable exogenous or heterologous nucleic acid sequences encoding a protein with FabD activity may also include derivatives of the nucleic acid sequence shown above, which include sequences that code for proteins or polypeptides having FabD activity with at least 50% identity or similarity (e.g., at least 60%, at least 70%, at least 80%, at least 90%, at least 95%, at least 96%, at least 97%, at least 98%, or at least 99%) at the amino acid level with the following sequence: MKFAFVFPGQGSQSVGMLNAFADNAVVRATVEEASAALGQDLGRLIAEGPAEELNL TTNTQPVMLTAAVAVYRAWLDAGGPAPALVAGHSLGEYSALVAAGVIPFADAVPL VRFRAQAMQEAVPVGEGAMAAILGLSDDDVRAACAEASAAGVVEAVNFNAPSQV VIAGHKAAVEKACEIAKGKGAKRALPLPVSAPFHSSLLKPASDRLRERMAGLAFSAP SIPLVNNVDVAIVNDPDAIKDALVRQAAAPVRWVECVQKMAAEGVTHVIECGPGKV LAGMTKRIDGNLTGGAIFDPASLQDTLALLK. Suitable exogenous or heterologous nucleic acid sequences encoding a protein with FabD activity may also include nucleic acid sequences which can be derived as a result of the degenerate genetic code from the amino acid sequence identified above as well as functional equivalents with FabD activity.

Suitable exogenous or heterologous nucleic acid sequences that that encode a protein with FabB activity include, but are not limited to, a nucleic acid sequence with the sequence shown below:

ACCGCGGTGGCGGCCGCTCTAGAACTAGTGAATAATTTTGTTTAACTTT
AAGAAGGAGGTATATCCATGGCTAGCATGACTAAACATATTTTGAAAGC
ACGAGGGGAAATCTAATGTCACCGCTGCTGTTCTCGCATTTCACGGCGA
CCAGCTGCCTGGGTGCGGGCGTGGACGCCACGCTGTCGGCACTGCGCGC
ACAGCGCGGCGGCCTGGCACCCTGCCGCTTCGGCGGCGTGGAACTCGAC
ACTTTCGTCGGCGAAGTGCCCGGCCTGGATACCGTGGCGCTTCCCCCGG
CGCTGGCCGAGTTCGACTGCCGCAACAACCGCCTGGCCCAGCTCGCGCT
GGAACAGGACGGTTTCGCAGCGCGGGTGCGCGAGGCGGCCGCCCGCTAC
GGTGCGCACCGGGTCGGCGTGTTCCTGGGCACTTCGACCGCAGGCGTGC
TCCAGACCGAACTGGCCTACCGCCAGCGCGATCCCGCCTCCGGAGCGCT
GCCGCCCGGCTTCCACTACGCAACCACGCACAACCCGTACAGCCTGCCC
GCCTTCCTGCGCCAGTACCTGGGCCTGACTGGCCCCGCCGCCGCGGTGT
CCTCAGCCTGCTCGTCCGGAGCGAAGGTGTTCTCGTCCGCGCGGCGCAT
GCTGGAAGCGGGCCTGATCGACGTGGCCGTGGTGGGCGGCGTCGATTCG
CTGTGCCATACCACGCTGTACGGCTTCAACAGCCTGGAGCTGCTGTCGG
ATCAGCCGTGCCGTCCGTACGACGTGGCGCGCAACGGCATCTCGATCGG
CGAAGGTGCCGCCTTCGGCCTGCTGGAGCGTGTGGCAGGCAGCGCCCAG
GGCGATGCGATCCTGCTGGCGGGCATCGGCGAATCCAGCGATGCGCACC
ACATGTCCACGCCGCACCCGGACGGCCTGGGTGCGCGCCTGGCACTGGA
ACAGGCGCTGGCCTCGGCCGGTGTCGCCCCCGCCCAGGTCGGCTACGTC
AACCTGCATGGTACCGCCACGCGCAGCAACGACGCCGCCGAAGCCATGG
CTATGGCCGCCGTGCTGCCGGGCACGCCCTGCTCGTCGACCAAGGGTGC
GACCGGCCATGCGCTCGGTGCCGCCGGGGCGCTTGAAGCGGTGATCTGC
GCGCTGGCGCTGCGCCACGGCCTGATGCCCGGCGGCATCAATACTACCC
AGGTCGATCCGGCGCTGGACGTCAACTACCAGCTGGCCAACCGCGAGGC
ACCGCTGCGCTACGCCATGACCAACGCATTTGGCTTCGGCGGCAGCAAC
TGCTCCCTGCTGTTCGCGCGTGCCGACGCCGCCAGCCATTAGCCCCGGG
CTGCAGGAATTCGATATCAAGCT

Suitable exogenous or heterologous nucleic acid sequences encoding a protein with FabB activity may also include derivatives of the nucleic acid sequence shown above, which include sequences that code for proteins or polypeptides having FabB activity with at least 50% identity or similarity (e.g., at least 60%, at least 70%, at least 80%, at least 90%, at least 95%, at least 96%, at least 97%, at least 98%, or at least 99%) at the amino acid level with the following sequence: MSPLLFSHFTATSCLGAGVDATLSALRAQRGGLAPCRFGGVELDTFVGEVPGLDTVA LPPALAEFDCRNNRLAQLALEQDGFAARVREAAARYGAHRVGVFLGTSTAGVLQTE LAYRQRDPASGALPPGFHYATTHNPYSLPAFLRQYLGLTGPAAAVSSACSSGAKVFS SARRMLEAGLIDVAVVGGVDSLCHTTLYGFNSLELLSDQPCRPYDVARNGISIGEGA AFGLLERVAGSAQGDAILLAGIGESSDAHHMSTPHPDGLGARLALEQALASAGVAP AQVGYVNLHGTATRSNDAAEAMAMAAVLPGTPCSSTKGATGHALGAAGALEAVIC ALALRHGLMPGGINTTQVDPALDVNYQLANREAPLRYAMTNAFGFGGSNCSLLFAR ADAASH. Suitable exogenous or heterologous nucleic acid sequences encoding a protein with FabB activity may also include nucleic acid sequences which can be derived as a result of the degenerate genetic code from the amino acid sequence identified above as well as functional equivalents with FabB activity.

Suitable exogenous or heterologous nucleic acid sequences that that encode a protein with FabH activity include, but are not limited to, a nucleic acid sequence with the sequence shown below:

ATGACCAAATACGCAAAAATCATCGGTACCGGCAGCTACCTGCCGCCGC
GCCGTGTGACCAACCACGATCTGGCGACCCAGCTGGCCGAGAAGGGCAT
CGAGACTTCCGACGACTGGATCGTGTCGCGTTCGGGCATCAGCGCGCGG
CACTGGGCCGAGCCGGACGTGACCTCCAGCGATCTGGCCGTCAAGGCGG
CCGAACAGGCCATCGAGGCTGCTGGCATCGATCGCCAGTCGATCGATCT
GATCATCGTGGCCACTTCGACGCCGGACTTTGTCTTCCCCTCGACCGCC
TGCATCGTCCAGGAGAAACTTGGCATTACCAACCACTGCCCCGCCTTCG
ATCTGCAAGCCGTATGCTCGGGCTTCGTCTACGCCCTGGCCACGGCCGA
CAAGTTTATCCGCTCCGGCTCGCACCGCAACGTGCTGGTCATCGGTACC
GAGGTCTTCTCGCGCATCCTGGACTTCAACGATCGCACGACCTGCGTGC
TGTTCGGGGACGGTGCCGGTGCGGTGCTGCTGAGCGCCTCCGAGGAACC
CGGCATCCTGTCGACCGCCATGCATTCCGACGGCCGCCACGTCGACATC
CTGTGCGTGCCGGGCAACGTTGCAGGCGGCAATATTACCGGCAACCCGT
TCCTGCACATGGACGGCCAGGCGGTGTTCAAGCTGGCCGTCAATGTGCT
CGACAAGGTGGCCCGCGAGGCGATGGAAGCCGCCTCGGTGACGCCGGGC
CAGATCGACTGGCTGATCCCGCACCAGGCCAACATCCGCATCATGCAGG
GTACCGCCAAGAAGCTCGGCCTGCCCGCCGAACGCATGGTCGCCACGGT
GCACGAGCACGGCAATACCTCAGCCGCCTCGATCCCGCTGGCGCTGGAC
GTGGCCGTGCGCGACGGCCGCATCCGTGCGGGCCAGACCGTGCTGATGG
AAGGGGTCGGCGGCGGCTTTACCTGGGGTGCCGTGCTGCTGCGCATGTA
A

Suitable exogenous or heterologous nucleic acid sequences encoding a protein with FabH activity may also include derivatives of the nucleic acid sequence shown above, which include sequences that code for proteins or polypeptides having FabH activity with at least 50% identity or similarity (e.g., at least 60%, at least 70%, at least 80%, at least 90%, at least 95%, at least 96%, at least 97%, at least 98%, or at least 99%) at the amino acid level with the following sequence: MTKYAKIIGTGSYLPPRRVTNHDLATQLAEKGIETSDDWIVSRSGISARHWAEPDVTS SDLAVKAAEQAIEAAGIDRQSIDLIIVATSTPDFVFPSTACIVQEKLGITNHCPAFDLQA VCSGFVYALATADKFIRSGSHRNVLVIGTEVFSRILDFNDRTTCVLFGDGAGAVLLSA SEEPGILSTAMHSDGRHVDILCVPGNVAGGNITGNPFLHMDGQAVFKLAVNVLDKV AREAMEAASVTPGQIDWLIPHQANIRIMQGTAKKLGLPAERMVATVHEHGNTSAASI PLALDVAVRDGRIRAGQTVLMEGVGGGFTWGAVLLRM. Suitable exogenous or heterologous nucleic acid sequences encoding a protein with FabH activity may also include nucleic acid sequences which can be derived as a result of the degenerate genetic code from the amino acid sequence identified above as well as functional equivalents with FabH activity.

An engineered strain of C. necator can express a Fab enzyme alone or in combination with one or more of Fab. Additionally, an engineered strain of C. necator can express a desaturase enzyme alone or in combination with one or both of FabB, FabD, FabF, FabH, or FabZ.

Other proteins or enzymes involved in fatty acid biosynthesis or lipid metabolism that can recombinantly or heterologously expressed in the engineered C. necator of the present disclosure include, but are not limited to, acyl-CoA dehydrogenases, acyl-ACP [=acyl carrier protein] desaturases, acyl-ACP thioesterases, fatty acid-re-acyl transferases, acyl-CoA: lysophospholipid acyl transferases, fatty acid synthases, fatty acid hydroxylases, acetyl-coenzyme A carboxylases, acyl coenzyme A-oxidases, fatty acid desaturases, fatty acid acetylenases, lipoxygenases, triacylglycerol lipases, allen oxide synthases, hydroperoxide lyases, and fatty acid elongases.

Other proteins or enzymes involved in fatty acid biosynthesis include omega 3 polyunsaturated fatty acid synthases. Suitable exogenous or heterologous nucleic acid sequences encoding a protein with omega 3 polyunsaturated fatty acid synthase activity may include sequences that code for proteins or polypeptides having omega 3 polyunsaturated fatty acid synthase activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Omega-3 polyunsaturated fatty acid synthase subunit PfaA:

MSHTPSQPQPSTDKKADKRLNKRLKDMPIAIVGMASIFANSRYLNKFWDLIC
DKIDAITDVPASHWAIDDYYDVDKSKADKSYCKRGGFMPEVDENPMEFGLPPNILEL
TDSSQLLSLVVAKEVLQDANLPDDYDRDRIGITLGIGGGQKLSHSLNARLQYPVLKK
VFKSSGLSDEDSELLIKKFQDQYVHWEENSFPGSLGNVIAGRIANRFDLGGMNCVVD
AACAGSLAAMRMALTELTEGRSDMMITGGVCTDNSPYMYMSFSKTPAFTTNEQIQP
FDIDSKGMMIGEGIGMVALKRLDDAERDGDRIYAVIKGVGASSDGKFKSIYAPRPEG
QAKALERAYDDAGFAPHTVGLIEAHGTGTAAGDVAEFTGLSSVFSQDNAQLQHIAL
GSVKSQVGHTKSTAGTAGVIKAALALHHKVLPPTINVSKPNPKLEIDRSPFYLNTEAR
PWIQRSDDTPRRAGISSFGFGGTNFHLVLEEYRPDHTRDDAYRQRSVAQILLFAAND
KTLLLNELKAVLQQASSAKAELSEAHFIQFAKPYALREITPQSARLGFIAKDYAQLQT
LLTQAIAQLEANNAESWQLPSGISYRAKALVNEQTKIAALFAGQGAQYLNMGLELA
NNFPELRRHIHASDKVFSTHGKPALSSVLYPIPAFDDESIKAQETALTNTLYAQSAIGA
LSMAQYALFTQAGFAPDMLAGHSFGELSALCAAGVISMDDYIKLAFERGQAMAQSS
QDTDAGVMYAVILKQKQDIEVINGCLAQFEGVKVANYNSPTQLVIAGASAATQQAA
KAISELGFKAIALPVSGAFHTPLVAHAQKPFSAAIDKAQFNTPKIALYANGTGQLHPI
DANAIKAALKDHMLQSVHFSEQLEAMYAAGARVFVEFGPKNILQKLTENTLAAQLN
ELCIISINPNPKGDSDSQLRSAAVQLAVAGVKLREIDPYQAELIAPAATSAMNIKLNAT
NYISPATRSKMVDSLQSGKITSQVQYVDRIVEKVVEKVVEKPVIVEKILEKVVEVEKP
VAQNSNNIQQQTPAQPASFTAGQTNQDALSAFFAAQTQAAQLHQQFLAIPQQYGDT
VSALMAEQAKMASLGIAIPESLQRSMELFHQHQAQTLKSHSDFMQLQTSSSQAVLAL
LGQMPASQVQAPIQAAAPVAVAVTKPVVPAQAPVVQGLAAEPKVTAVPVSEPTVQ
QPQVALAQVAQTKVTQPPLAQPQVQTVAAQTSALQVKPALQQIEHAMLSVVADKT
GYPVEMLELSMDMEADLGIDSIKRVEILGTVQDELPNLPELSPEDLAECRTLGEIVAL
FSQAAPVTSATTVSHATQSAVAASAAVSNDEIERTMMAVVADKTGYPVEMLELSM
DMEADLGIDSIKRVEILGTVQDELPNLPELSPEDLAECRTLGEIVALFSQAVPVAAQTF
AAMAATNPQVVASAVTPIAAVSDGEIEHTMMAVVADKTGYPVEMLELSMDMEAD
LGIDSIKRVEILGTVQDKLPNLPELSPEDLAECRTLGEIAALFSQAAPVTAAATVSHAT
QSAIAARAAVSNDEIERTMMAVVADKTGYPVEMLELSMDMEADLGIDSIKRVEILG
TVQDQLPNLPELSPEDLAECRTLGEIVALYAGSQSSSEALQQNHAATIQETQEAIAKT
VEETIDLPPHSEVMLKKLPAAAELARIIATSDVQLTANSYVVIGDDGHNAGVIAEKLH
AQGVKVAVVRSPKTVVTSASPLDSHIASFTLEAIDDESICEVINQIEALGQIAGFIHLQP
QHKSVADKGAGLVLVDEAKASVEQAFLFAKFLQPLLTERDYCRFVTVSCIDGGFGYI
GMDESVGALISQSELNQAALFGLTKTLNHEWPGVVCRALDIAPNLDAKTVANAVVQ
EYYLQDAPVEVGIDSNFDRVTLVAGTAALRHPPAVLSNADKILVTGGAKGVTFECA
LSLAKRCQAHFFLAGRSAHQVIPAWAEGKKSNELKAAAIAHLQSLGDKPTPKQVDA
LVWPVQSSLEISHALAAFDAIGASAEYLSVDVNDPAAIASTIAPINALSPITGIIHGAGV
LADKHIQDKTLNEFERVYGTKVTGLNNLLSTLDLSQVKLIALFSSAAGFYGNTGQSD
YAMSNDILNKAALQLAQQLPQAKVMSFDWGPWDGGMVNPALKKMFIDRGVYVIP
LKAGAELFASQLLSDTGAQLLVGTDMQGNTANAVEVASAKKPEADLATALDPQPM
AQTVPQSIRVMRSLDPKRMSFIEDHCINGHAVLPTVCAIDWMREAAKAHLGTAVSV
SDYRLLKGVIFDEALLARNAPIELELMLTPLADAAQQSTEALAALISFEGRPQYQAVL
VAQTDDMPDAQRFEVGELHSLIQEMAQQPAIANRESLYSDGTLFHGPRLQGISEVLT
FDDQHLMAKVELPQVALDDCGKFAPKLEDKGTQPFAEDLLLQAMLVWARLKYQA
ASLPSTIGEFVSYAPLSFGEKAVLVLDVLKHSSRSLEANIALYHQDGRLSCEMKRAK
VTISKTLNQAFLANKPQQLAQVQASIQNMAEVSVK

Omega-3 polyunsaturated fatty acid synthase PfaB:

MTLNPIAHAALSNAALNSPHPNAKPLRIAVWLGDGWMQSALSHAQTDASAL
QQLMACRIQLNVEDKLLIDEPSDEPSDESTLVALLSEQLAHIAQKQLVEIRFEYQQQV
RSLFLLDGLLAAQLHLHAEAYISALAQTQAEANEALCDIEIENCTNRAFALAKRDCA
QAVNCYSDAGNLASQLKLLYQAIEALSHRTLAGITPMLAHLNTEKTERCYWFSKPH
QARVLSLNLFDKAPQAQTAQSLILTQGTGLIAQPLLNANRLFIPISGNEFESLTLKLLQ
LIDSLTLSLNQPDTDWLSSQGSDWFKRYQAKDELALVLMAGSLEELMQEAKAMQTF
IEKARLTIESSASKHSASKPSASTSLVFKTPAGSYFAASPLGDKGLTFVYPGVGTVYPN
MFSDLHSYFPELYRELEREGDLAAMLQAETIYQDAAYAKTAVNVSVKDTAEMSLSQ
LAISGVGASYLFSKLLTGVFTIQPRLALGYSMGEAAMWASLAIWQTPHSLIDATQGS
AIFNHEISGKLQAVRRDWQLNEDAPLAWNSFLVRATSTEINPLLADFPRVYLAIEQG
DTCILAGCEASCLQLLARLNKRGIASNKVTAMHTAPSQSQRNAIQGFYTLGLKATAC
ETQVRFISAAQHSPVNIDSMSIAKSIADTFCAPLNFTALINTAYNQGARLFVEVGADR
QTSTLIDKISRQLELGADGVQEPILAMACNAKGSDTIVSLLKCLAQLISHRVPLSLAAL
MPQSAAQSATHSATIHADKTAAKTIASHSANACALGHYSNVFQEGEPL

Omega-3 polyunsaturated fatty acid synthase PfaC:

MSSQMHTHPTLQDSAAVPNDQRQTLKAMPKIAIVGLAVQYPDADTPEQFWQ
NLLDKKDSRSQIDAAKLNANPADYQGIQGQADRFYCDKGGYIRNFRFDPQGYQLLP
ATFAGLDESFLWALDCSKKALLNAGVDLTAPLLERTGIVMGTLSFPTARSNELFLPIY
HQAVEKALKTKLNQPQFALAPFANASIAGSQLAANGVIAHTASKLLSDALGLGGAQ
LSLDAACASSVYALKLACDYLTTGKADMMLAGAVSGADPFFINMGFSIFHAYPDHG
ISAPFDSNSKGLFAGEGAGVLVLKRLEDAERDGDNIYAVVSGIGLSNDGKGQFVLSP
NSKGQVQAFERAYAAANTHPSNIEVIECHATGTPLGDKVELTSMERFFEDKLDGTKA
PLIGSAKSNLGHLLTAAGMPGIMKMIFAMRSGHLPPSINLTAPISSPKGLFSVNNLPTQ
RQAWPDKAGNDRRHAGVSVFGFGGCNAHLLLESYQPTAHSAEKQANKPVYQQQA
LTVIGMASHFGPLASINALDKALIAQTDAFIPLPPKRWKGLDKHPDILQQFGLNRAPK
GAYIEQFDFDFLRFKVPPNEDDRLISQQLLLIKVADEAIRDAKLTAGSKVAVLVAMET
ELELHQFRGRVNLHTQLADSLKKQGVHLSNDEYLALEAIAMDSVLDAAKLNQYTSF
IGNIMASRIASLWDFNGPAFTISAAEQSVARCIDVAQNLLSKEALDGVVIAAVDLSGS
VEQVILKNAQVAVDLDANSANPQWKVGEGAGAIVLTNQQASNSQQAGYGQIRGQA
FGTNHQLPKLLDSLITETAIANPSMPTAIHMIEQCIAPEEQLPAEHLLAQLNLLGTSCN
RVANTLGHNFAAAGMASLLSALLSLKNRSANSDKNAEKQALVSTQSQGVSSLLLLS
QTATQAAQLELRLAQDLTLSEQKHLIKPVTLGGRDIYQHIVDTPLPALAAIQGKMRQ
LQPLASQATQTKPAVGAALDITAENATPLAAESGMSSNAPLQFETTASAQDSAALLQ
NQQLAREAHLAFLQSREQGLKLADALLKAQLSQTTQMGAVAAHVATSANVAETKA
QQAVSIPELMPNHAPNHARVPPYTPPIPAAKPCIWNYQDLVEYAEGDIAKVFGADYA
IIDSYARRVRLPTSDYLLVSRVTKLNAQMNRYQPSSMTTEYDIPVDAPFLVDGQIPW
AVAVESGQCDLMLISYLGIDFENKGERVYRLLDCTLTFLGDLPRGGDTLRYDISINHF
ARNGDTLLFFFSYECFVGDKLILKMDGGCAGFFTDKELADGKGVIRTEVEIKVREQA
QIALANEYTRNGNKPRFTPLLNCAQTAFSYGQIHRLLSADIGGCFGGEHAAHQAKFG
LQPSLCFASEKFLMIEQVSKLEVHGGAWGLGLIEGHKQLAPDHWYFPCHFKGDQVM
AGSLMAEGCGQLLQFFMLHIGMHANTQAGGVTNGRFQPLENASQKVRCRGQVLPQ
SGTLTYRMEVTEIGMSPRPYAKANIDILLNGKVVVDFQNLGVMIKEEADCTRYSQSH
SSQGNHTQAANIESLAEQAPLMAQIPDVAAPVNKGVVPLKHVSAPIAPAGSKYANR
VPDTLPFTPYHLFEFATGDIENCFGPDFSIYRGLIPPRTPCGDLQLTTRVVAIEGKRGEL
KKPSTCIAEYEVPSNAWYYRKTSHPSVMPYSVLMEISLQPNGFISGYMGTTLGFPGQ
ELFFRNLDGSGKLLREVDLRGKTIVNDSRLLSTVIAGSNIIQNFSFELSCDGEPFYRGN
AVFGYFKADALKNQLGIDNGKITQAWHLERGIKADCQINLLDKNGRSFVAPLGKPH
YRLAGGQLNFIDKAEIVKTGGKKGLGYLYAERTIDPSDWFFQFHFHQDPVMPGSLG
VEAIIELLQTYAIDQDLGAGFNNPKFGQILSEIKWKYRGQINPLNKQMSLDVHITSIED
KDGKRIIKGDANLSKDGLRIYEVTDIAICIEEA

Omega-3 polyunsaturated fatty acid synthase PfaD:

MTNTTLDNNALDNNKLSPWPWQVDEAAISFDIESLGKKLKDLNQACYLINH
AEKGLGIAQSAEVVGLAEPNNGLHPVSAFAPALGTQSLGDSNFRRVHGVKYAYYAG
AMANGIASEELVIALGQAGILCSFGAAGLIPSRVEAAIKRIQAALPNGPYAFNLIHSPS
EQALERGSVELFLKHQVRTVEASAFLGLTPQIVYYRAAGLSRDASGEIVIGNKVIAKI
SRTEVATKFMEPAPVKILQQLVNEGLISEDQMLMAQSVPMADDITAEADSGGHTDN
RPLVTLLPTILALKDTIQAKYQYKTPIRVGAGGGIGTPDAALATFNMGAAYIVTGSIN
QACVEAGASEHTRKLLATTEMADVTMAPAADMFEMGVKLQVVKRGTLFPMRANK
LYEIYTRYDSIEAIPAEERQKLEEQVFRASLDEIWAGTVAHFNERDPKQIERALDNPK
RKMALIFRWYLGLSSRWSNTGEVGREMDYQIWAGPALGAFNAWAKGSYLDDYRE
RNAVDLAKHLMQGAAYQARINLLLSQGVSIPVSLQRWKPLQRC.

Heterologous proteins involved in fatty acid biosynthesis and the transgenes that express them can be derived from a variety of organisms that natively produce desirable fatty acids, such as certain bacteria, cyanobacteria, fungi, algae, and other microbes or animals.

Thus, heterologous elongases or proteins involved in fatty acid biosynthesis, as well as nucleic acid sequences encoded the same, can be derived from a cyanobacteria such as a cyanobacteria from the genera Synechococcus. The Synechococcus species can be selected from Synechococcus ambiguus Skuja, Synechococcus arcuatus, Synechococcus bigranulatus, Synechococcus brunneolus, Synechococcus caldarius, Synechococcus capitatus.

Synechococcus carcerarius, Synechococcus elongatus, Synechococcus endogloeicus, Synechococcus epigloeicus, Synechococcus ferrunginosus, Synechococcus intermedius, Synechococcus koidzumii, Synechococcus lividus, Synechococcus marinus, Synechococcus minutissimus, Synechococcus mundulus, Synechococcus nidulans, Synechococcus rayssae, Synechococcus rhodobaktron, Synechococcus roseo-persicinus, Synechococcus roseo-purpureus, Synechococcus salinarum, Synechococcus salinus, Synechococcus sciophilus, Synechococcus sigmoideus, Synechococcus spongiarum, Synechococcus subsalsus, Synechococcus sulphuricus, Synechococcus vantieghemii, Synechococcus violaceus, Synechococcus viridissimus, and Synechococcus vulcanus. In some embodiment, the protein or nucleic acid is derived from Synechococcus elongatus.

Additionally or alternatively, the heterologous elongases or proteins involved in fatty acid biosynthesis, as well as nucleic acid sequences encoded the same, can be derived from a bacteria such as E. coli, a Sterptomyces sp., a Psuedomonas sp., a Clostridia sp., or a Bacillus sp.

Additionally or alternatively, the heterologous elongases or proteins involved in fatty acid biosynthesis, as well as nucleic acid sequences encoded the same, can be derived from algae, for example algae from the Prasinophyceae family, such as from the genera Heteromastix, Mammella, Mantoniella, Micromonas, Nephroselmis, Ostreococcus, Prasinocladus, Prasinococcus, Pseudoscourfielda, Pycnocetraselis, and Pycnococas. Particular species include, but are not limited to Heteromastix longifillis, Mamiella gilva, Mantoniella squamata, Micromonas pusilla, Nephroselmis olivacea, Nephroselmis pyriformis, Nephroselmis rotunda, Ostreococcus tauri, Ostreococcus sp. Prasinocladus ascus, Prasinocladus lubricus, Pycnococcus provasolii, Pyramimonas amylifera, Pyramimonas disomata, Pyramimonas obovata, Pyramimonas orientalis, Pyramimonas parkeae, Pyramimonas spinifera, Pyramimonas sp., Tetraselmis apiculata, Tetraselmas Tetraselmis Tisetraselmis Tetraselmis Tisetraselmis Tetraselmis Tisetraselmis Tetraselmis Tis hazeni, Tetraselmis impellucida, Tetraselmis inconspicua, Tetraselmis levis, Tetraselmis maculata, Tetraselmis marina, Tetraselmis striata, Tetraselmis subcordiformis, Tetraselmis suecica, Tetraselmis tetrabrachia, Tetraselmisetrosa rubens, or Tetraselmis sp. Additionally contemplated are algae of the Euglenaceae family such as from the genera Ascoglena, Astasia, Colacium, Cyclidiopsis, Euglena, Euglenopsis, Hyalophacus, Khawkinea, Lepocinclis, Phacus, Strombomonas, or Trachelomonas Particular species include, but are not limited to, Euglena acus, Euglena genicaoclenatais, Euglena geniculatais Euglena rostrifera, Euglena viridis, Colacium stentorium, Trachelomonas cylindrica or Trachelomonas volvocina.

### C. Expression of Transgenes

The nucleic acid sequences (e.g., transgenes) that are transfected or otherwise incorporated into the engineered C. necator of the present disclosure can be incorporated into an expression cassette. In doing so, the nucleic acid sequences that encode a Δ9-desaturase, a Δ12-desaturase, a Δ15-desaturase, a Δ6-desaturase, a Δ5-desaturase, and a Δ4-desaturase, or the nucleic acid sequence that encode Δ5-elongases, Δ6-elongases and Δ9-elongases, or the nucleic acid sequences encoding DesA, DesB, DesC, FabA, FabB, FabD, FabF, FabH, or FabZ can be operably linked with one or more regulatory signals for initiating, inducing, or enhancing gene expression. These regulatory sequences are intended to make possible the specific expression of the genes and proteins and may be referred to as a “gene construct.” The gene construct may also comprise one or more enhancer sequences in operable linkage with the promoter, which make possible an enhanced expression of the nucleic acid sequence. Additional advantageous sequences, such as further regulatory elements or terminator sequences, may also be inserted at the 3′ end of the DNA sequences. The transgenes may be present in one or more copies of the expression cassette (or gene construct).

The regulatory sequences or factors preferably have a positive influence on the gene expression of the introduced genes and thereby increase expression. The regulatory elements can advantageously be strengthened at the transcription level by using strong transcription signals such as promoters and/or enhancers.

For the purposes of the present disclosure, an expression cassette or expression vector may comprise one transgene encoding a desaturase, elongase, or other enzyme involved in fatty acid biosynthesis. Alternatively, the expression cassette or expression vector may comprise multiple transgenes encoding a combination of desaturases, elongases, or other enzymes involved in fatty acid biosynthesis

Various methods can be used to introduce an expression vector into the C. necator. Such methods are generally described in Sambrook et ah, Molecular Cloning: A Laboratory Manual, Cold Springs Harbor Laboratory, New York (1989, 1992), in Ausubel et ah, Current Protocols in Molecular Biology, John Wiley and Sons, Baltimore, Md. (1989), Chang et ah, Somatic Gene Therapy, CRC Press, Ann Arbor, Mich. (1995), Vega et ah, Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, and infection with recombinant viral vectors. Stable expression of transgenes can also be achieved, for example, using CRISPR-based systems, TALON, or zinc finger nucleases.

### VII. SINGLE CELL PROTEIN AND USES THEREOF

The present disclosure provides edible products, food, and others products, such as single cell protein products, that include protein derived from the engineered C. necator bacterium provided herein. Additionally provided are methods for producing amino acids, proteins, fatty acids, oils, and other biological nutrients using the engineered C. necator described herein.

Current single cell protein (SCP) products are largely lacking in desirable unsaturated fatty acids, including ω-3 and ω-6 fatty acids. The engineered C. necator disclosed herein can be utilized in methods of preparing a SCP product that includes desirable unsaturated fatty acids and does not require further fortification. The methods may comprise culturing an engineered strain of C. necator, thereby creating biomass comprising protein and unsaturated fatty acid(s). The methods may also comprise isolating the C. necator or the biomass, and preparing a single-cell protein product from the C. necator or the biomass. The protein-rich biomass may be used as a single cell protein (SCP) product. The biomass may be utilized as an edible product, for instance, an animal feed or human food.

In some embodiments, the animal feed is a fish feed, a livestock or ruminant feed, a swine feed, goat feed, llama feed, turkey feed, a poultry feed, a rodent feed, a dog feed, and a cat feed. The animal feed may be a livestock feed or a domesticated animal feed. In yet other aspects, the human food is a yogurt, a smoothie, a bread product, a pasta product, a nutritional bar, a chip or cracker, a plant-based meat substitute, a cheese, a plant-based cheese, and a powdered nutritional supplement.

One of the major challenges in utilizing biosystems for food production is obtaining the proper dietary balance between the quantities of protein, carbohydrate, and fat. The microbial systems generally considered for food synthesis tend to produce biomass disproportionately high in protein and saturated fatty acids. The engineered C. necator strains of the present disclosure can be used to produce a SCP product with a higher proportion of fats and oils, particularly, unsaturated fatty acids. Thus, the present disclosure provides food products, including human foods and animal feeds comprising a SCP produced from or including the engineered C. necator described herein.

A SCP product, “protein product,” or “microbial protein product” (e.g., one or more of single cell protein, cell lysate, protein concentrate, protein isolate, protein extract, protein hydrolysate, free amino acids, peptides, oligopeptides, or combinations thereof), derived from one or more engineered C. necator strains described herein, may be processed or incorporated into an edible food composition for human and/or animal consumption, a cosmetic, a pharmaceutical product, or a fertilizer.

A food composition (i.e., food product) may be, for example, a food item, a food ingredient, a nutritional product, an animal feed, and/or a pet food product. In some embodiments, the food composition may contain any of at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A cosmetic or pharmaceutical composition may contain any of at least about 0.01%, at least about 0.05%, at least about 0.1%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A fertilizer may contain any of at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

The biomass that is produced from culturing the engineered C. necator strains disclosed herein results in a desirable protein and fatty acid content. The biomass can have a protein content higher than or at least about any of about 40%, about 50%, about 60%, about 70%, about 80%, about 90%, or about 95% by weight, and a fat content of about 60%, about 50%, about 40%, about 30%, about 20%, about 15%, about 10%, or about 5% by weight.

The biomass or an SCP product created therefrom may comprise free amino acids, polypeptides, proteins, oils, lipids, and/or fatty acids. In some embodiments, an SCP product derived from the engineered C. necator strains disclosed herein comprises an increased unsaturated and/or polyunsaturated fatty acid content relative to an SCP product derived from a wild type or naturally occurring strain of C. necator. For instance, the SCP product comprises about 0.25 wt % to about 25 wt %, about 0.5 wt % to about 25 wt %, about 1 wt % to about 25 wt %, about 5 wt % to about 25 wt %, about 10 wt % to about 25 wt %, about 15 wt % to about 25 wt %, about 20 wt % to about 25 wt %, about 0.5 wt % to about 20 wt %, about 1 wt % to about 20 wt %, about 5 wt % to about 20 wt %, about 10 wt % to about 20 wt %, about 15 wt % to about 20 wt %, about 0.5 wt % to about 15 wt %, about 1 wt % to about 15 wt %, about 5 wt % to about 15 wt %, about 10 wt % to about 15 wt %, about 0.5 wt % to about 10 wt %, about 1 wt % to about 10 wt %, about 5 wt % to about 10 wt %, about 0.5 wt % to about 5 wt %, or about 1 wt % to about 5 wt % of polyunsaturated fatty acids. Additionally or alternatively, the SCP product comprises about 0.25 wt % to about 25 wt %, about 0.5 wt % to about 25 wt %, about 1 wt % to about 25 wt %, about 5 wt % to about 25 wt %, about 10 wt % to about 25 wt %, about 15 wt % to about 25 wt %, about 20 wt % to about 25 wt %, about 0.5 wt % to about 20 wt %, about 1 wt % to about 20 wt %, about 5 wt % to about 20 wt %, about 10 wt % to about 20 wt %, about 15 wt % to about 20 wt %, about 0.5 wt % to about 15 wt %, about 1 wt % to about 15 wt %, about 5 wt % to about 15 wt %, about 10 wt % to about 15 wt %, about 0.5 wt % to about 10 wt %, about 1 wt % to about 10 wt %, about 5 wt % to about 10 wt %, about 0.5 wt % to about 5 wt %, or about 1 wt % to about 5 wt % of unsaturated fatty acids. Additionally or alternatively, the SCP product comprises about 0.25 wt % to about 25 wt %, about 0.5 wt % to about 25 wt %, about 1 wt % to about 25 wt %, about 5 wt % to about 25 wt %, about 10 wt % to about 25 wt %, about 15 wt % to about 25 wt %, about 20 wt % to about 25 wt %, about 0.5 wt % to about 20 wt %, about 1 wt % to about 20 wt %, about 5 wt % to about 20 wt %, about 10 wt % to about 20 wt %, about 15 wt % to about 20 wt %, about 0.5 wt % to about 15 wt %, about 1 wt % to about 15 wt %, about 5 wt % to about 15 wt %, about 10 wt % to about 15 wt %, about 0.5 wt % to about 10 wt %, about 1 wt % to about 10 wt %, about 5 wt % to about 10 wt %, about 0.5 wt % to about 5 wt %, or about 1 wt % to about 5 wt % of unsaturated fatty acids and/or polyunsaturated fatty acids.

Compositions comprising the disclosed SCP product can find application in various industries such as food, pharmaceutical, nutraceutical, cosmetic, agriculture, consumer goods, construction etc. The compositions can be used as food agents for human or animal consumption, cosmetic agents, pharmaceutical agents, nutritional agents, fertilizer and other agricultural agents, industrial agents, or any combination thereof. Illustrative applications include animal feed, food and beverages, infant formula, toddler formula, special dietary needs formula, reduced allergenicity formulas, skin-care and hair-care compositions, fertilizers, etc.

In some aspects, the SCP products as described herein are utilized in the production of a vegetarian or vegan food product. In certain embodiments, the SCP products are utilized in the production of an organic food product and/or pesticide-free and/or herbicide-free and/or fungicide-free and/or antibiotic-free food product. The SCP products may be utilized in a probiotic food product or in a prebiotic food product. In some aspects the SCP product may not include animal protein or fats.

In some embodiments, the SCP product can be incorporated into food products including, but not limited to, dairy products, dairy replacement products, meat products (including livestock, game, poultry, fish, or seafood products), meat replacement and/or imitation meat products (including imitation livestock, game, poultry, fish, or seafood products), bakery products, confections, health and protein bars, protein powders, sports and/or energy drinks, and/or protein shakes and/or smoothies. The type of food and beverage is not particularly limited, and can include, for example, noodles, instant noodles, soups, instant soups, pasta, microwave foods, canned foods, freeze-dried foods, soft drinks, fruit juice drinks, vegetable drinks, infant formula, toddler formula, non-dairy milk, coffee drinks, tea drinks, nutritional beverages, powdered beverages, protein powders, nutritional supplements, concentrated beverages, alcoholic beverages, breads, cake mixes, rice cakes, flour products, chewing gum, gummies, chocolate, caramel, cookies, snacks, chips, pretzels, crackers, biscuits, cakes, pies, confectionery, sauces, processed seasonings, flavor seasonings, cooking mixes, curries, stews, sauces, dressings, oils and fats, butter, margarine, mayonnaise and other condiments, milk drinks, yogurt, lactic acid bacteria drinks, ice creams, cream processed fish products, processed livestock products, agricultural canned products, jams and marmalades, pickles, cereals, nutritional foods, vegan or vegetarian meat substitutes, and the like. In certain embodiments, protein products are textured for incorporation into meat products and/or imitation meat products.

In some aspects, the SCP product has a high ratio of unsaturated fats to saturated fats. In certain embodiments, the SCP product may have an amino acid content is substantially similar, very close, or identical to that recommended by the United Nations Food and Agriculture Organization as “ideal.” In certain embodiments, food products made using the SCP products of the present disclosure represent healthy and/or low-calorie foods. The SCP product may be formed into fibers and/or thermally extruded and/or coagulated into a gel. Gel coagulation occurs at pH falling in a range of about 3 to about 6 upon heating. In some aspects, one or more properties of the SCP product makes it well suited for incorporation into food products, including but not limited to dairy products, dairy replacement products, meat products, meat replacement and/or imitation meat products, bakery products, confections, health and protein bars, protein powders, sports and/or energy drinks, and/or protein shakes and/or smoothies. The SCP product may be used as a meat extender, for example, as a meat extender in food products. In some aspects, water absorption and/or fat binding properties of the SCP product aids in reducing shrinkage (fat and water loss) on cooking and/or enhances the moisture and texture of the cooked patty or other meat or food item.

The SCP product may impart improved nutrition, water absorption, fat binding properties, texture, and/or eating qualities to a food product, such as a cereal based product. The SCP product may be used to fortify or is otherwise incorporated into a cereal based product. The cereal based product may be a breakfast cereal, cookie, cake, pie, brownie, muffin, or bread. In some aspects, the protein product may be used as a replacement for milk proteins (e.g. sodium caseinate) and/or as a vitamin and/or mineral supplement in milk or dairy products. The protein SCP ingredient may be used in one or more of non-fat dried milk, powdered milk, or dairy type drinks, such as, but not limited to, instant breakfast mixes, or imitation dairy type drinks including but not limited to soy milk, rice milk, and almond milk. The SCP product ingredient may be used in nutritionally fortified (e.g., protein, vitamin, and/or mineral fortified) candies, deserts, or treats.

The SCP product may be processed to produce a food product or ingredient thereof, in a process that includes heating the protein product, optionally in combination with other ingredients, optionally under shearing agitation, followed by extrusion to produce a product of desired texture (e.g., chewy, crunchy, crispy, resists dispersion in water, etc.). The SCP product can be processed to produce a food product or ingredient thereof, in a process that includes combining the SCP product with one or more additional protein sources (including, but not limited to, pea, rice, glutinous rice, wheat, gluten, soy, hemp, canola, insects, algae, and/or buckwheat).

In some aspects, free amino acids are included, either as part of the SCP product or supplemental to the SCP product, to impart a desired flavor. In one non-limiting embodiment, glutamic acid is included, thereby imparting an umami flavor to the food product.

In some aspects, for example, in a meat substitute or artificial meat product, a hydrogel, lipogel, and/or emulsion can be combined the SCP product, for example, as an agent release system (e.g., for release of a coloring agent, a flavor agent, a fatty acid, a leavening agent, a gelling agent (e.g., bicarbonate (e.g., potassium bicarbonate), calcium hydroxide, and/or alginate (e.g., sodium or potassium alginate)), wherein the agent(s) may be released during cooking of the food product to simulate animal meat).

In some aspects, a food product comprising the SCP produce may also include one or more plant protein source such as, but not limited to, pea, rice, glutinous rice, wheat, gluten, soy, hemp, canola, insects, algae, and/or buckwheat, in combination with a protein product produced by microorganisms as described herein (e.g., one or more of single cell protein, cell lysate, protein concentrate, protein isolate, protein extract, protein hydrolysate, free amino acids, peptides, oligopeptides, or combinations thereof), wherein the protein product imparts a flavor to the food composition, such as, for example, a meat-like flavor (including a livestock, game, poultry, or seafood meat-like flavor).

In some aspects, a food product, for example, a meat substitute or artificial meat product, includes a heme compound, such as a heme-containing polypeptide. In one embodiment, the food product includes heme (e.g., heme-containing polypeptide) from the microorganism from which the protein product is derived.

A meat substitute or artificial or imitation meat product (e.g., a livestock (e.g., beef, pork), game, poultry, fish, or seafood analogue product) may include a protein product produced by microorganisms as described herein (e.g., one or more of single cell protein, cell lysate, protein concentrate, protein isolate, protein extract, protein hydrolysate, free amino acids, peptides, oligopeptides, or combinations thereof). In some embodiments, the meat analogue product is a vegan product that does not contain any ingredients from animal sources. In some embodiments, an enhanced meat product which contains animal protein (e.g., a beef, poultry, pork, fish, seafood, or egg product) and comprises a protein product ingredient produced by microorganisms as described herein (e.g., one or more of single cell protein, cell lysate, protein concentrate, protein isolate, protein extract, protein hydrolysate, free amino acids, peptides, oligopeptides, or combinations thereof)), is provided. For example, the protein product may be included as an extender in an enhanced meat product or in a meat analogue product, e.g., the SCP product replaces any of at least about 10%, at least about 20%, at least about 30%, at least about 40%, at least about 50%, at least about 60%, or at least about 70% of the meat ingredient or an artificial or imitation meat ingredient (for example, a plant-based artificial or imitation meat analogue ingredient) to produce the enhanced meat product or meat analogue/imitation meat product, respectively.

In some aspects, the protein product is used as an aquaculture feed or in an aquaculture feed formulation. In some embodiments, the protein-rich biomass is used as a high-protein substitute for fishmeal used in aquaculture and/or other animal feed products. The animal feed may include up to 20% (w/w) or up to 10% (w/w) SCP product, wherein the SCP product comprises engineered C. necator cells described herein.

Protein and/or biomass produced according to the present disclosure may be converted to animal feed using methods and processes well known in the art and science of chemistry, chemical engineering, and food science. The feed produced through the disclosure may be used to grow organisms including but not limited to one or more of the following: other microorganisms, yeast, fungi, zooplankton, shellfish (e.g., shrimp, prawns, crabs, scallops, clams, mussels, etc.) or other invertebrates, fish, birds, and mammals. In certain non-limiting embodiments, the fish include but are not limited to one or more of: tilapia, tuna, salmon, cod, cobia, and haddock. The birds may include, but are not limited, to chickens, pheasants, or turkeys. The mammals may include but are not limited to one or more of: rodents, rabbits, goats, sheep, pigs, cows, horses, deer, dogs, cats, buffalo, llamas, alpacas, non-human primates, and aquatic mammals (e.g., dolphins, whales, manatees, etc.). The feed may be used to grow live-feed that in turn sustain finfish larvae through the first weeks of life. The feed produced may be used to grow zooplankton organisms including but not limited to one or more of the following: rotifers [Phylum Rotifera]; order Cladoceran (e.g., Daphnia sp., Moina sp.); sub-class Copepoda (e.g., Cyclops); Brine shrimp (Anemia sp.).

In some embodiments, the animal feed comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the animal feed comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the animal feed comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

The microbial cells of the present disclosure may be boiled prior to feeding to another organism (e.g., human or animal). The cells may sonicated, or otherwise lysed or ruptured prior to feeding to another organism (e.g., human or animal).

In addition to consumable or edible food products (e.g., human foods and animal feed), SCP products or biomass produced by or derived from the engineered C. necator disclosed herein can also be used as a fertilizer. The fertilizer may be applied to crop plants, ornamentals, turf grass, or aquacultures (e.g., algae or seaweed).

In some embodiments, the fertilizer comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the fertilizer comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the fertilizer comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

Starting with wet or dry microbial biomass produced as described herein, in certain embodiments, protein may be concentrated using a process comprising one or more of the following steps: liquid-solid extraction, removal and recovery of a solvent from the liquid extract, removal and recovery of a solvent from the solid (e.g., the protein concentrate), and drying and grinding of the solid (e.g., the protein concentrate).

A solid-liquid extraction may be performed batchwise or continuously. The solid-liquid extraction may be performed using one or more of: horizontal belt extractors; basket extractors; stationary extractors; and/or rotary cell extractors.

A non-polar solvent may be utilized in a solvent extraction step. A non-polar solvent may be utilized in combination with an alcohol solvent. A non-polar solvent may be utilized in combination with an aqueous alcohol solution. A non-polar solvent can be utilized to extract neutral lipids from an extract produced using alcohol and/or an aqueous alcohol solution. The non-polar solvent may be utilized that has a boiling point range (i.e., distillation range) of 65° C. to 70° C. A non-polar solvent may be utilized that consists primarily of six-carbon alkanes. In certain aspects, hexane is utilized as a non-polar solvent. The hexane utilized as a non-polar solvent may comply with the strict quality specifications required for the extraction of edible oils from soybean and other plant-based sources, including but not limited to: boiling (distillation) range, maximum non-volatile residue, flash point, maximum sulfur, maximum cyclic hydrocarbons, color and specific gravity. In certain aspects, “supercritical extraction” using liquid carbon dioxide under high pressure is utilized for solvent extraction.

The cell mass, i.e., microbial biomass produced as described herein may be kept in liquid suspension when subjected to solvent extraction or if dried, may be fed as a loose power with open, porous structure into a solvent extraction process. The rate of extraction can be increased by applying one or more of agitation and/or increasing the temperature. Higher temperature can result in higher solubility of the extractable material (e.g., lipid), and/or higher diffusion coefficients.

Water-free (absolute) low aliphatic alcohols, such as ethanol or isopropanol, are suitable solvents for lipids at high temperature, but the solubility of oils in these solvents decreases drastically as the temperature is lowered. In certain embodiments, lipid extraction takes place at high temperature one or more alcohol, including but not limited to ethanol, isopropanol, and/or methanol. In certain such embodiments, the lipid extract is cooled, and lipid saturation occurs. In certain such embodiments, the excess lipid separates as a distinct phase, which can be recovered by a solid-liquid separation process, such as, but not limited to, centrifugation. In certain such embodiments, the solvent, i.e., alcohol(s), is reheated and sent back for solvent extraction.

When a concentration gradient is used to transfer the extractable substance out of a solid, keeping the gradient high can facilitate the extraction process. In certain embodiments, the principle of counter-current multistage extraction is utilized to exploit this effect. In certain embodiments, the solvent extraction process is divided into a number of contact stages. In certain embodiments, each stage comprises the mixing of solid, e.g., microbial biomass and/or protein concentrate, and the solvent phases, and the separation of the two streams after extraction is achieved. In certain embodiments, in going from one stage to the next, the solids, e.g., microbial biomass and/or protein concentrate, and the solvent flow in opposite directions. Thus, microbial biomass and/or protein concentrate with the lowest extractable content (e.g., lipids) are contacted with the leanest solvent, resulting in higher extractable yield (e.g., lipid yield) and high driving force throughout the extractor.

The cell culture may be harvested in a logarithmic phase and/or in an arithmetic phase and/or in a stationary phase. Extraction can be performed using batch, semi-continuous and/or continuous solvent extractors.

In batch processes, a certain quantity of microbial biomass and/or biological material is contacted with a certain volume of fresh solvent. In certain embodiments, the extract is drained off, distilled and the solvent is recirculated through the extractor until the residual extractable content (e.g., lipid content) in the batch of microbial biomass and/or biological material is reduced to a targeted level.

A semi-continuous solvent extraction system may utilize that consists of several batch extractors connected in series. In certain such embodiments, the solvent and/or extract flows from one extractor to the next one in the series. In certain non-limiting embodiments, a French Stationary Basket Extractor is utilized.

A continuous solvent extraction process may be utilized in which microbial biomass and/or biological material and/or protein concentrate and solvent are fed continuously into an extractor.

A protein product (e.g., one or more of single cell protein, cell lysate, protein concentrate, protein isolate, protein extract, protein hydrolysate, free amino acids, peptides, oligopeptides, or combinations thereof), is derived from and/or includes biomass and/or protein isolate, protein extract, protein hydrolysate, free amino acids, peptides, and/or oligopeptides derived from one or more engineered C. necator strains described herein and may be produced by any method described herein. The disclosed protein products provide the additional benefit of including desirable unsaturated and polyunsaturated fatty acids, which means the products can be prepared without separate or additional fortification with fatty acids.

### VIII. PRODUCTION OF UNSATURATED FATTY ACIDS

Provided herein are lipid composition prepared from engineered C. necator biomass, wherein the lipid composition comprises one or more unsaturated or polyunsaturated fatty acids produced by the engineered C. necator. Also provided herein are methods for preparing a lipid composition comprising one or more fatty acids produced by the engineered C. necator strains disclosed herein. The methods may include obtaining the lipid composition from a C. necator biomass, wherein the biomass comprises the one or more unsaturated fatty acids or polyunsaturated fatty acids (PUFAs).

The unsaturated fatty acid compositions of the present disclosure may include a C16 fatty acid, a C18 fatty acid, or both a C16 fatty acid and a C18 fatty acid. The C16 fatty acid may include a palmitoleic acid, a sapienic acid, and any combination thereof. The C18 may include an oleic acid, a linoleic acid, an α-linoleic acid, a γ-linolenic acid, a linolelaidic acid, an elaidic acid, a vaccenic acid, a stearidonic acid, and any combination thereof. The structure of said fatty acids may include:

Methods for obtaining a lipid composition from the biomass include, but are not limited to, extraction, heat, pressure, saponification, sonication, freezing, grinding, ion exchange, chromatography, membrane separation, electrodialysis, reverse osmosis, distillation, chemical derivatization, crystallization, etc.

For example, cellular lipids comprising the one or more unsaturated fatty acids can be extracted from the biomass by any suitable method including, but not limited to, extraction with a solvent including, but not limited to, ethanol, ethyl acetate, isopropyl alcohol, methanol, ethyl acetate, hexane, methylene chloride, methanol, chloroform, and the like, or by pressurized liquid hydrocarbons such as butane, pentane, propane, or others (with our without co-solvents), or through supercritical fluid extraction (with or without co-solvents). The extracted oil can be evaporated under reduced pressure to reduce or remove the solvent and/or produce a sample of concentrated lipid material.

The cells can be broken or lysed to obtain the lipid composition, for example into vegetable or other edible oil. Extracted oils can be subjected to refining (e.g. chemical refining, physical refining). A refining process can remove some or all impurities from extracted oils. The refining process can comprise one or more methods to degum, bleach, filter, deodorize and/or polish the extracted oils.

The one or more unsaturated fatty acids contained in the extracted lipid composition can be concentrated by hydrolyzing the lipids and concentrating an unsaturated fatty acids fraction, preferably an unsaturated fatty acids fraction, by employing a method such as, for example, urea adduction, fractional distillation, column chromatography, and/or supercritical fluid fractionation.

The unsaturated fatty acids can be extracted from the biomass to provide the lipid composition using a solvent under an extraction condition sufficient to extract unsaturated fatty acids and/or molecules comprising unsaturated fatty acids but not compounds that are insoluble in the solvent. In some aspects, the unsaturated fatty acids can be extracted by separating the cellular debris and precipitated compounds using a separation method such as filtration, centrifugation, and/or combinations thereof.

The solvent may be a polar solvent. Examples of polar solvent include, but are not limited to, ethanol, ethyl acetate, isopropyl alcohol, methanol, ethyl acetate, and mixtures thereof. In one aspects, the polar solvent is ethanol.

Extraction of the lipid composition with a solvent can be carried out in a variety of ways. For example, the extraction can be a batch process, a continuous process, or a continuous counter-current process. In a continuous counter-current process, the solvent contact with the microalgae leaches the oil into the solvent, providing increasingly more concentrated miscellas (i.e., solvent-oil), while the solvent-solids is contacted with miscellas of decreasing concentration. Following extraction, the solvent can be removed from the miscella using methods known in the art. For example, distillation, rotary evaporation, or a rising film evaporator and steam stripper or any suitable desolventizer can be used for removing the solvent.

The extracted lipid (i.e., the lipid composition) may be subjected to an absorption process (e.g., bleaching) to remove one or more undesirable compounds such as, for example, color bodies and/or phosphatides that may be present. For example, the absorption process can be a bleaching process comprising contacting the lipid composition with a bleaching material (e.g., neutral earth (also termed natural clay or fuller's earth), acid-activated earth, activated carbon, activated clays, silicates, and or a combination thereof) and a filter aid. Precipitates that form after acid mixing can be removed from the lipid composition, for example using centrifugation and/or filtration (e.g., membrane filtration).

The extracted lipid may additionally be subjected to a degumming step. Degumming methods are known in the art including, for example, water degumming, acid degumming, enzymatic degumming, and membrane degumming. The degumming step may include contacting the lipid composition with a mixture of aqueous acids that are in amounts effective to precipitate gums and/or chlorophyll-type compounds that may be present in the lipid composition. The degummed lipid composition can be subjected to drying for a time, a temperature, and/or a vacuum condition sufficient to reduce a moisture content of the composition. In some embodiments, the moisture content of the dried lipid composition is less than about 10 weight percent, illustratively, less than 10, 5, 1, 0.9, 0.8, 0.7, 0.6, 0.5, 0.4, 0.3, 0.2, 0.1, 0.05, ad 0.01 weight percent.

The fatty acid esters with polyunsaturated C16 fatty acid and a C18 fatty acid molecules can be isolated in the form of an oil or lipid, for example in the form of compounds such as sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides or other fatty acid esters such as the acetyl-coenzyme A esters which comprise the polyunsaturated fatty acids with at least two, three, four, five or six, preferably five or six double bonds, from the organisms which have been used for the preparation of the fatty acid esters; preferably, they are isolated in the form of their diacylglycerides, triacylglycerides and/or in the form of phosphatidylcholine, especially preferably in the form of the triacylglycerides. In addition to these esters, the polyunsaturated fatty acids are also present in the organisms, advantageously in the bacterial cell as free fatty acids or bound in other compounds.

The unsaturated fatty acids produced can be isolated from the organisms by methods known to those skilled in the art, as described above. For example, via extraction, distillation, crystallization, salt precipitation and/or chromatography, if necessary. For this purpose, the organisms can be advantageously digested beforehand.

The one or more unsaturated fatty acids can be provided in any one of variety of forms/compositions suitable for a particular application or use. Thus, for example, in some embodiments, a whole-cell microalgae biomass and/or a fraction and/or extract thereof, provides the one or more unsaturated fatty acids. In another embodiment, the unsaturated fatty acids are in a powdered form or as a free oil in a liquid form (e.g., lipid composition or a fraction or concentrate thereof).

In various embodiments, human and/or animal consumption/use of the one or more unsaturated fatty acids is contemplated. Accordingly, in one embodiment, the unsaturated fatty acids produced by the microalgae is provided in a form and/or grade suitable for use in an end-product selected from the group consisting of: a feed, a dietary supplement, a food, a pharmaceutical formulation, a dairy product, and an infant formula.

The unsaturated fatty acid composition may include unsaturated fatty acids at a concentration in a range from 0.01% to 100%. The amount of the fatty acid composition in any of the food products described herein can be between 0.01% and 99.99% by weight of the food product. Thus, the amount of fatty acid composition in the food product can be 0.1%, 0.2%, 0.3%, 0.4%, 0.5%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, 99.7%, 99.8%, 99.9% and the like. In other embodiments, the amount of the fatty acid composition in the food product is in a range from 0.1% to 5%, 0.1% to 10%, 0.1% to 15%, 0.1% to 20%, 0.1% to 25%, 0.1% to 30%, 0.1% to 35%, 0.1% to 40%, 0.1% to 45%, 0.1% to 50%, 0.1% to 60%, 0.1% to 70%, 0.1% to 80%, 0.1% to 90%, 0.1% to 99%, 0.1% to 99.5%, 0.5% to 5%, 0.5% to 10%, 0.5% to 15%, 0.5% to 20%, 0.5% to 25%, 0.5 to 35%, 0.5% to 45%, 0.5% to 55%, 0.5% to 65%, 5% to 25%, 5% to 35%, 5% to 45%, 5% to 55%, 5% to 65%, 5% to 75%, 5% to 80%, 5% to 85%, 5% to 95%, 5% to 99%, 10% to 30%, 10% to 40% 10% to 50%, 10% to 60%, 10% to 70%, 10% to 75%, 10% to 80%, 10% to 85%, 10% to 95%, 10% to 99%, 10% to 99.9%, 15% to 35%, 15% to 45%, 15% to 55%, 15% to 65%, 15% to 75%, 15% to 85%, 15% to 95%, 15% to 99%, 15% to 99.9%, 20% to 40%, 20% to 50%, 20% to 60%, 20% to 70%, 20% to 75%, 20% to 80%, 20% to 85%, 20% to 95%, 20% to 99%, 25% to 40%, 25% to 50%, 25% to 60%, 25% to 70%, 25% to 75%, 25% to 80%, 25% to 85%, 25% to 95%, 25% to 99%, 30% to 50%, 30% to 55%, 30% to 60%, 30% to 65%, 30% to 70%, 30% to 75%, 30% to 80%, 30% to 85%, 30% to 90%, 30% to 95%, 30% to 99%, 35% to 50%, 35% to 55%, 35% to 60%, 35% to 65%, 35% to 70%, 35% to 75%, 35% to 80%, 35% to 85%, 35% to 90%, 35% to 95%, 35% to 99%, 40% to 50%, 40% to 55%, 40% to 60%, 40% to 65%, 40% to 70%, 40% to 75%, 40% to 80%, 40% to 85%, 40% to 90%, 40% to 95%, 40% to 99%, 45% to 60%, 45% to 65%, 45% to 70%, 45% to 75%, 45% to 80%, 45% to 85%, 45% to 90%, 45% to 95%, 45% to 99%, 50% to 60%, 50% to 65%, 50% to 70%, 50% to 75%, 50% to 80%, 50% to 85%, 50% to 90%, 50% to 95%, 50% to 99%, 55% to 65%, 55% to 70%, 55% to 75%, 55% to 80%, 55% to 85%, 55% to 90%, 55% to 95%, 55% to 99%, 60% to 70%, 60% to 75%, 60% to 80%, 60% to 85%, 60% to 90%, 60% to 95%, 60% to 99%, 65% to 80%, 65% to 85%, 65% to 90%, 65% to 95%, 65% to 99%, 70% to 80%, 70% to 85%, 70% to 90%, 70% to 95%, 70% to 99%, 75% to 85%, 75% to 90%, 75% to 95%, 75% to 99%, 80% to 90%, 80% to 95%, 80% to 99%, 85% to 90%, 85% to 95%, 85% to 99%, 90% to 95%, 90% to 99%, 95% to 99%, and the like.

### IX. EXAMPLES OF EMBODIMENTS

The following non-limiting list of embodiments provides examples of particular implementations of the present disclosure. Those skilled in the art will understand that additional and alternative embodiments may also be disclosed here.

Embodiment 1. A strain of Cupriavidus necator, comprising a transgene encoding a heterologous desaturase enzyme, wherein the strain of C. necator synthesizes at least one unsaturated C18 fatty acid that is not natively produced by C. necator.

Embodiment 2. The strain of C. necator of embodiment 1, wherein the at least one unsaturated C18 fatty acid is selected from an oleic acid, a linoleic acid, an α-linoleic acid, a γ-linolenic acid, a linolelaidic acid, an elaidic acid, a vaccenic acid, a stearidonic acid, and any combination thereof.

Embodiment 3. A strain of C. necator that expresses a heterologous desaturase enzyme encoded by a transgene, wherein the strain of C. necator synthesizes an increased amount of an unsaturated C16 and/or C18 fatty acid relative to a strain of C. necator that does not express the heterologous desaturase enzyme.

Embodiment 4. The strain of C. necator of embodiment 3, wherein the unsaturated fatty acid is a C16 fatty acid, a C18 fatty acid, or both a C16 fatty acid and a C18 fatty acid.

Embodiment 5. The strain of C. necator of embodiment 4, wherein the C16 fatty acid is selected from a palmitoleic acid, a sapienic acid, and any combination thereof.

Embodiment 6. The strain of C. necator of embodiments 4 or 5, wherein the C18 fatty acid is selected from an oleic acid, a linoleic acid, an α-linoleic acid, a γ-linolenic acid, a linolelaidic acid, an elaidic acid, a vaccenic acid, a stearidonic acid, and any combination thereof.

Embodiment 7. The strain of C. necator of any one of embodiments 1-6, wherein the transgene is codon optimized for expression in Cupriavidus necator.

Embodiment 8. The strain of C. necator of any one of embodiments 1-7, wherein the strain of C. necator is modified, optionally by adaptive laboratory evolution or genetic modification.

Embodiment 9. The strain of C. necator of any one of embodiments 1-8, wherein the strain of C. necator grows autotrophically at up to 40° C.

Embodiment 10. The strain of C. necator of embodiments 8 or 9, wherein the strain of C. necator has a H₂:CO₂ uptake ratio that is lower than the H₂:CO₂ uptake ratio of a wild type or naturally occurring strain of C. necator cultured under corresponding temperatures and conditions.

Embodiment 11. The strain of C. necator of embodiment 10, wherein the strain of C. necator comprises a full or partial deletion of a gene or locus encoding a membrane-bound hydrogenase.

Embodiment 12. The strain of C. necator of embodiment 11, wherein the membrane-bound hydrogenase is hoxKGXZ.

Embodiment 13. The strain of C. necator of any one of embodiments 1-12, wherein the strain of C. necator produces about 40%, about 50%, about 60%, about 70%, about 80%, about 90%, or about 95% by weight of protein.

Embodiment 14. The strain of C. necator of any one of embodiments 1-13, wherein the heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 15. The strain of C. necator of any one of embodiments 1-14, wherein the heterologous desaturase enzyme is from a cyanobacteria or a eukaryote.

Embodiment 16. The strain of C. necator of embodiment 15, wherein the cyanobacteria is a Synechococcus species (sp).

Embodiment 17. The strain of C. necator of any one of embodiments 1-16, wherein the strain of C. necator further comprises a second transgene encoding a second heterologous desaturase enzyme.

Embodiment 18. The strain of C. necator of embodiment 17, wherein the second heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 19. The strain of C. necator of embodiments 17 or 18, wherein the second heterologous desaturase enzyme is from a cyanobacteria or a eukaryote.

Embodiment 20. The strain of C. necator of any one of embodiments 17-19, wherein the strain of C. necator further comprises a third transgene encoding a third heterologous desaturase enzyme.

Embodiment 21. The strain of C. necator of embodiment 20, wherein the third heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 22. The strain of C. necator of embodiments 20 or 21, wherein the third heterologous desaturase enzyme is from a cyanobacteria or a eukaryote.

Embodiment 23. The strain of C. necator of any one of embodiments 1-22, wherein the strain of C. necator expresses at least one of DesC, DesA, or DesB from a Synechococcus sp.

Embodiment 24. The strain of C. necator of any one of embodiments 1-23, wherein the strain of C. necator expresses DesC, DesA, and DesB from a Synechococcus sp.

Embodiment 25. The strain of C. necator of any one of embodiments 1-24 further comprising a transgene encoding an enzyme to increase C18:0 fatty acid synthesis.

Embodiment 26. The strain of C. necator of any one of embodiments 1-25, wherein the strain of C. necator overexpresses at least one of FabD, FabB, or FabF.

Embodiment 27. The strain of C. necator of embodiment 26, wherein the strain of C. necator overexpresses FabD, FabB, and FabF.

Embodiment 28. The strain of C. necator of any one of embodiments 1-27, wherein the strain of C. necator overexpresses at least one of FabG or FabZ.

Embodiment 29. The strain of C. necator of embodiment 28, wherein the strain of C. necator overexpresses FabG and FabZ.

Embodiment 30. The strain of C. necator of any one of embodiments 25-29, wherein C18:0 fatty acid synthesis is increased at least 50% relative to a wild-type strain of C. necator.

Embodiment 31. The strain of C. necator of any one of embodiments 1-30, wherein the strain of C. necator is deposited at DSM34774.

Embodiment 32. A method of preparing a single-cell protein product, comprising:
(a) culturing the strain of C. necator of any one of embodiments 1-31, thereby creating biomass comprising protein and fatty acid,
(b) isolating the C. necator or the biomass, and
(c) preparing a single-cell protein product from the C. necator or the biomass.

Embodiment 33. A method of producing a fatty acid, comprising culturing the strain of C. necator of any one of embodiments 1-31 such that the fatty acid is produced.

Embodiment 34. The method of embodiments 32 or 33, wherein culturing comprising growing the strain of C. necator in a fermentation tank.

Embodiment 35. The method of embodiment 34, wherein the fermentation tank is a gas fermentation tank.

Embodiment 36. The method of any one of embodiments 32-35, wherein culturing comprising growing the strain of C. necator in autotrophic conditions.

Embodiment 37. The method of embodiment 36, wherein the autotrophic conditions comprise providing carbon monoxide (CO) or carbon dioxide (CO₂) as a carbon source.

Embodiment 38. The method of any one of embodiments 32-37, wherein culturing comprising growing the strain of C. necator at a temperature above 30° C. and up to 40° C.

Embodiment 39. The method of any one of embodiments 33-38 further comprising isolating the fatty acid.

Embodiment 40. The method of any one of embodiments 33-38 further comprising isolating the strain of C. necator.

Embodiment 41. A single-cell protein (SCP) composition, comprising the strain of C. necator of any one of embodiments 1-31 or biomass created therefrom.

Embodiment 42. The SCP composition of embodiment 41, wherein the composition comprises at least about 40%, about 50%, about 60%, about 70%, about 80%, about 90%, or about 95% by weight of protein.

Embodiment 43. The SCP composition of embodiments 41 or 42, wherein the composition comprises at least about 0.1%, about 0.5%, about 1%, about 2%, about 5%, about 10% or about 15% of a fatty acid component.

Embodiment 44. The SCP composition of embodiment 43, wherein the fatty acid component comprises an oleic acid, a linoleic acid, an α-linoleic acid, a γ-linolenic acid, a linolelaidic acid, an elaidic acid, a vaccenic acid, a stearidonic acid, or any combination thereof.

Embodiment 45. The SCP composition of any one of embodiments 41-44, wherein the composition is produced by culturing the strain of C. necator of any one of embodiments 1-31, thereby creating biomass comprising protein and fatty acid, and isolating the C. necator or the biomass.

Embodiment 46. An edible product, comprising (a) the strain of C. necator of any one of embodiments 1-31 or biomass created therefrom, or (b) the SCP composition of any one of embodiments 41-45.

Embodiment 47. The edible product of embodiment 46, wherein the edible product is an animal feed.

Embodiment 48. The edible product of embodiment 47, wherein the animal feed is selected from a fish feed, a livestock or ruminant feed, a swine feed, goat feed, llama feed, turkey feed, a poultry feed, a rodent feed, a dog feed, and a cat feed.

Embodiment 49. The edible product of embodiment 46, wherein the edible product is a human food.

Embodiment 50. The edible product of embodiment 49, wherein the human food is selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

Embodiment 51. A method of preparing an edible product, comprising mixing (a) the strain of C. necator of any one of embodiments 1-31 or biomass created therefrom, or (b) the SCP composition of any one of embodiments 41-45 with one or more edible ingredients.

Embodiment 52. A topical product, comprising a fatty acid produced by the method of any one of embodiments 33-38.

Embodiment 53. The topical product of embodiment 52, wherein the product is a lotion, balm, cream, or makeup.

Embodiment 54. A nutritive or consumable composition comprising a strain of C. necator described herein.

Embodiment 55. The composition according to embodiment 54, wherein the composition is or comprises a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, or a cereal or cercal product.

Embodiment 56. The composition according to any of embodiments 54 to 55, wherein the composition is incorporated into one or more articles, converted into one or more second products, end-user products, consumer products, or any combination thereof.

Embodiment 57. The composition according to any of embodiments 54 to 56, wherein the composition is incorporated into pet food or animal feed.

Embodiment 58: A recombinant C1-fixing microorganism comprising a transgene encoding a heterologous desaturase enzyme, wherein the microorganism synthesizes at least one unsaturated C16 and/or C18 fatty acid that is not natively produced by the microorganism.

Embodiment 59: A recombinant C1-fixing microorganism that expresses a heterologous desaturase enzyme encoded by a transgene, wherein the microorganism synthesizes an increased amount of an unsaturated C16 and/or C18 fatty acid relative to a strain of the microorganism that does not express the heterologous desaturase enzyme.

Embodiment 60: The recombinant C1-fixing microorganism of embodiments 58 or 59, wherein the microorganism is selected from the group consisting of Cupriavidus necator and Ralstonia eutropha.

Embodiment 61: The recombinant C1-fixing microorganism of any one of embodiments 58-60, wherein the microorganism does not natively produce polyhydroxyalkanoates (PHAs).

Embodiment 62: The microorganism of any one of embodiments 58-61, wherein the unsaturated fatty acid is a C16 fatty acid, a C18 fatty acid, or both a C16 fatty acid and a C18 fatty acid.

Embodiment 63: The microorganism of embodiment 62, wherein the C16 fatty acid is selected from a palmitoleic acid, a sapienic acid, and any combination thereof.

Embodiment 64: The microorganism of embodiment 62 or 63, wherein the C18 fatty acid is selected from an oleic acid, a linoleic acid, an α-linoleic acid, a γ-linolenic acid, a linolelaidic acid, an elaidic acid, a vaccenic acid, a stearidonic acid, and any combination thereof.

Embodiment 65: The microorganism of any one of embodiments 58-64, wherein the transgene is codon optimized for the microorganism.

Embodiment 66: The microorganism of any one of embodiments 58-65, wherein the microorganism is modified, optionally by adaptive laboratory evolution or genetic modification.

Embodiment 67: The microorganism of any one of embodiments 58-66, wherein the microorganism grows autotrophically at up to 40° C.

Embodiment 68: The microorganism of embodiment 66 or 67, wherein the microorganism has a H₂:CO₂ uptake ratio that is lower than the H₂:CO₂ uptake ratio of a wild type or naturally occurring strain of C. necator cultured under corresponding temperatures and conditions.

Embodiment 69: The microorganism of embodiment 68, wherein the microorganism comprises a full or partial deletion of a gene or locus encoding a membrane-bound hydrogenase.

Embodiment 70: The microorganism of embodiment 69, wherein the membrane-bound hydrogenase is hoxKGXZ.

Embodiment 71: The microorganism of any one of embodiments 58-70, wherein the microorganism produces about 40%, about 50%, about 60%, about 70%, about 80%, about 90%, or about 95% by weight of protein.

Embodiment 72: The microorganism of any one of embodiments 58-71, wherein the heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 73: The microorganism of any one of embodiments 58-72, wherein the heterologous desaturase enzyme is from a cyanobacteria or a eukaryote.

Embodiment 74: The microorganism of embodiment 73, wherein the cyanobacteria is a Synechococcus species (sp).

Embodiment 75: The microorganism of any one of embodiments 58-74, wherein the microorganism further comprises a second transgene encoding a second heterologous desaturase enzyme.

Embodiment 76: The microorganism of embodiment 75, wherein the second heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 77: The microorganism of embodiment 75 or 76, wherein the second heterologous desaturase enzyme is from a cyanobacteria or a eukaryote.

Embodiment 78: The microorganism of any one of embodiments 75-77, wherein the microorganism further comprises a third transgene encoding a third heterologous desaturase enzyme.

Embodiment 79: The microorganism of embodiment 78, wherein the third heterologous desaturase enzyme is selected from a Δ9-desaturase, a Δ12-desaturase, a Δ15-desaturase, a Δ6-desaturase, a Δ5-desaturase, and a Δ4-desaturase.

Embodiment 80: The microorganism of embodiment 78 or 79, wherein the third heterologous desaturase enzyme is from a cyanobacteria or a eukaryote.

Embodiment 81: The microorganism of any one of embodiments 58-80, wherein the strain of C. necator expresses at least one of DesC, DesA, or DesB from a Synechococcus sp.

Embodiment 82: The microorganism of any one of embodiments 58-81, wherein the strain of C. necator expresses DesC, DesA, and DesB from a Synechococcus sp.

Embodiment 83: The microorganism of any one of embodiments 58-82 further comprising a transgene encoding an enzyme to increase C18:0 fatty acid synthesis.

Embodiment 84: The microorganism of any one of embodiments 58-83, wherein the microorganism overexpresses at least one of FabD, FabB, or FabF.

Embodiment 85: The microorganism of embodiment 84, wherein the microorganism overexpresses FabD, FabB, and FabF.

Embodiment 86: The microorganism of any one of embodiments 58-85, wherein the microorganism overexpresses at least one of FabG or FabZ.

Embodiment 87: The microorganism of embodiment 86, wherein the microorganism overexpresses FabG and FabZ.

Embodiment 88: The microorganism of any one of embodiments 83-87, wherein C18:0 fatty acid synthesis is increased at least 50% relative to a wild-type strain of the microorganism.

Embodiment 89: A method of culturing the microorganism according to any one of embodiments 58-89.

Embodiment 90: A product comprising the microorganism according to any one of embodiments 58-89 or a biological component (e.g., protein, fatty acid, etc.) derived from the microorganism.

### X. EXAMPLES

The following examples are given to illustrate the present disclosure. It should be understood, however, that the disclosure is not to be limited to the specific conditions or details described in these examples.

### Example 1: ALE Strain of Heat Resistant C. necator Bacteria

A continuous-flow stirred tank reactor (CSTR) was inoculated using a base strain of C. necator (H16 PHB-4) and grown continuously on minimal media and H₂/O₂/CO₂ inputs. The base strain, DSM 541, is a naturally occurring C. necator mutant that is unable to produce or express polyhydroxybutyrate (PHB). Once the culture was established and growing at steady-state, the reactor temperature was increased step-wise and held to allow the organism to adapt. These step changes were continued over ˜ 100 days and brought the reactor temperature from 30 to 39° C. Glycerol stocks were created and Whole Genome Sequencing conducted at various temperatures along the way to preserve progress and determine what beneficial mutations arose. Later, a separate CSTR was inoculated at 37° C. from the glycerol stock harvested at 37° C. during the evolution. This adapted strain started successfully, whereas other CSTRs using the base strain would only start at 30 and not 37° C., thus confirming the creation of a distinct phenotype.

As demonstrated in Table 1, the base strain was capable of growth on rich media during plate and inoculum growth at 30 to 37° C., but fails to grow autotrophically (minimal media, H₂/O₂/CO₂ feed) at 37° C. and above. However, the temperature evolved strain DSM 34774 surprisingly demonstrated growth under autotrophic conditions at 37° C. Further, DSM 34774 was capable of growth on CO₂/H₂/O₂ gas mixtures at elevated temperatures of up to 39° C.

| Run # | 1 | 2 | 3 | 4 | 5 |
|---|---|---|---|---|---|
| Strain | DSM | DSM | DSM | DSM | DSM |
| | 541 | 541 | 541 | 541 | 34774 |
| Plate growth [° C.] | 30 | 30 | 37 | 37 | 37 |
| Inoculum growth [° C.] | 30 | 30 | 37 | 37 | 37 |
| Reactor startup [° C.] | 30 | 37 | 37 | 30 | 37 |
| Successful startup? | Yes | No | No | Yes | Yes |

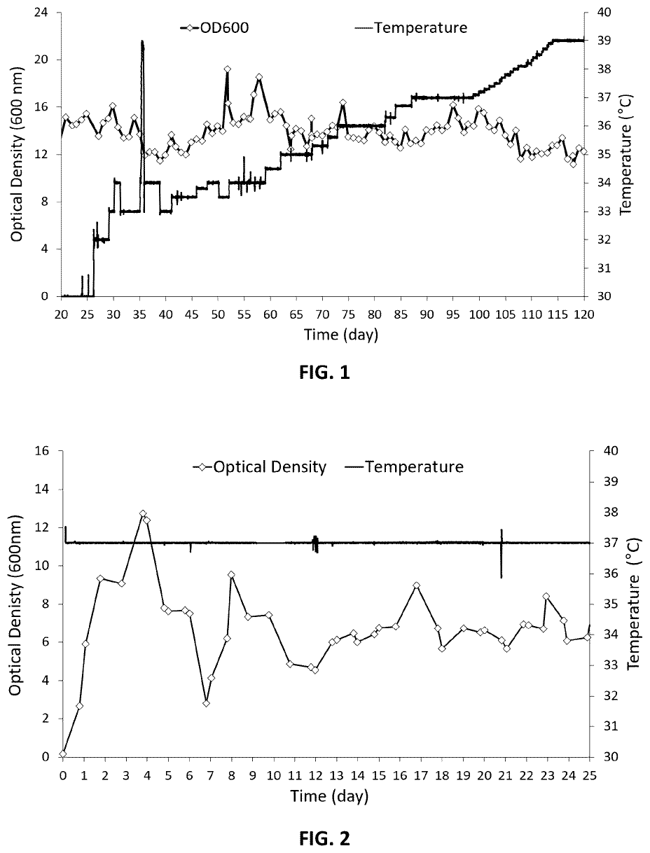

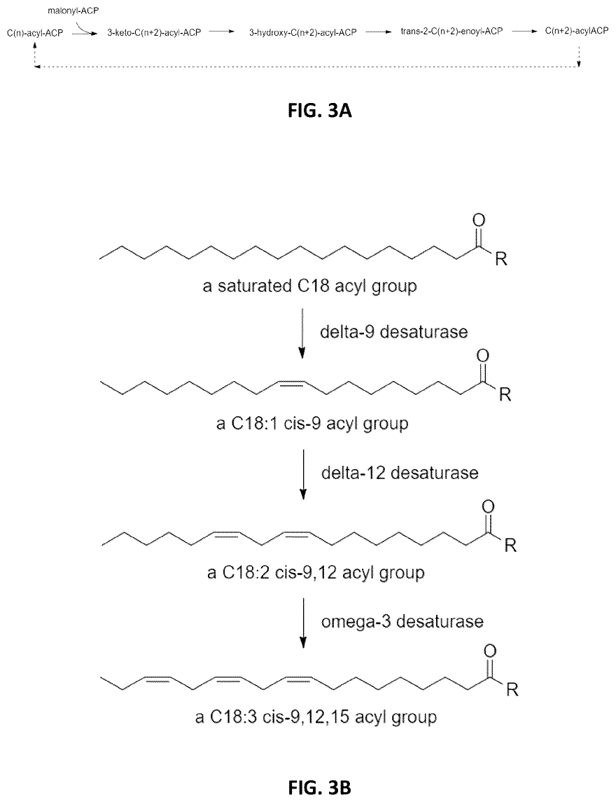

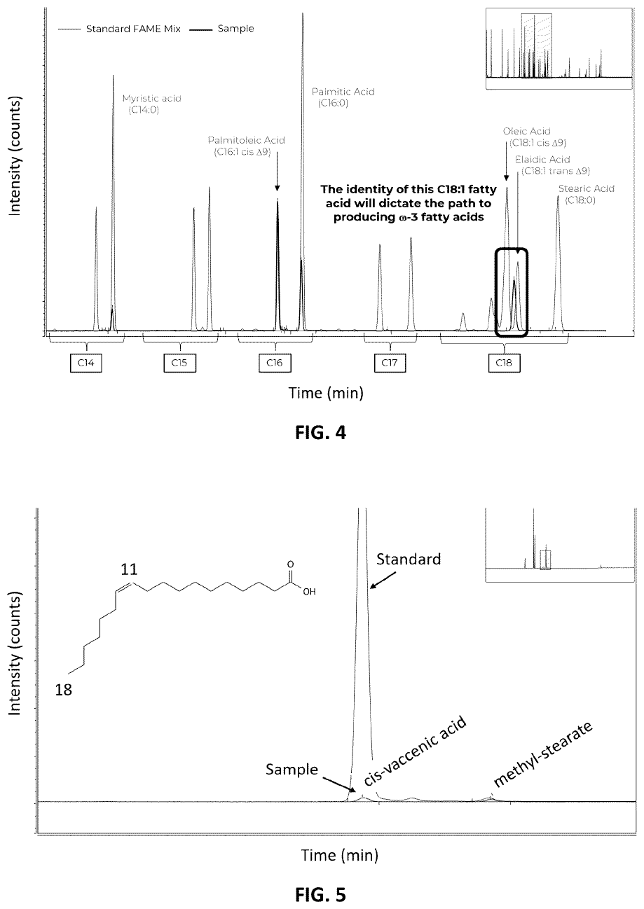

The evolved strain DSM 34744 was equally capable of sufficient culture and growth at lower temperatures ranging from 30-37° C. FIGS. 1 and 2 show biomass titers of this strain. FIG. 3A demonstrates native fatty acid biosynthesis pathway in C. necator with FabF catalyzing the initial chain elongation step. FIG. 3B demonstrates a heterologous pathway for biosynthesis of non-native unsaturated fatty acids in Cupriavidus necator.

### Example 2—C. necator Fatty Acid Profile

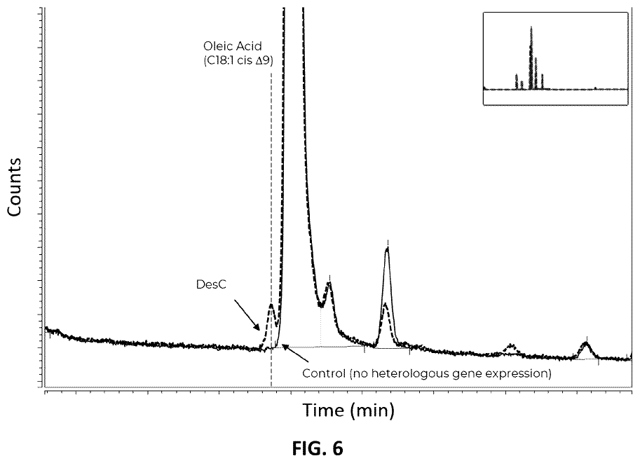

Applicant conducted GC-MS analysis on C. necator grown in continuous CO₂/H₂ culture systems. GCMS analysis indicates the presence of saturated and monounsaturated fatty acids, with minimal expression of polyunsaturated fatty acids. Further, GC-MS analysis demonstrated minimal saturated C18 fatty acids and C18Δ9 (FIG. 4).

As shown in FIG. 4, C. necator produces predominately C16 fatty acids as both C16:0 and C16:1 cis-Δ9. Importantly, C. necator produces only trace amounts of C18:0, which is a key precursors for C18 unsaturated fatty acids. Additionally, GC-MS analysis identified trace amounts of C12:0 and C: 14:0 likely corresponding to myristic acid. Trace amounts of C17 cyclopropane fatty acid were also indicated through GC-MS analysis.

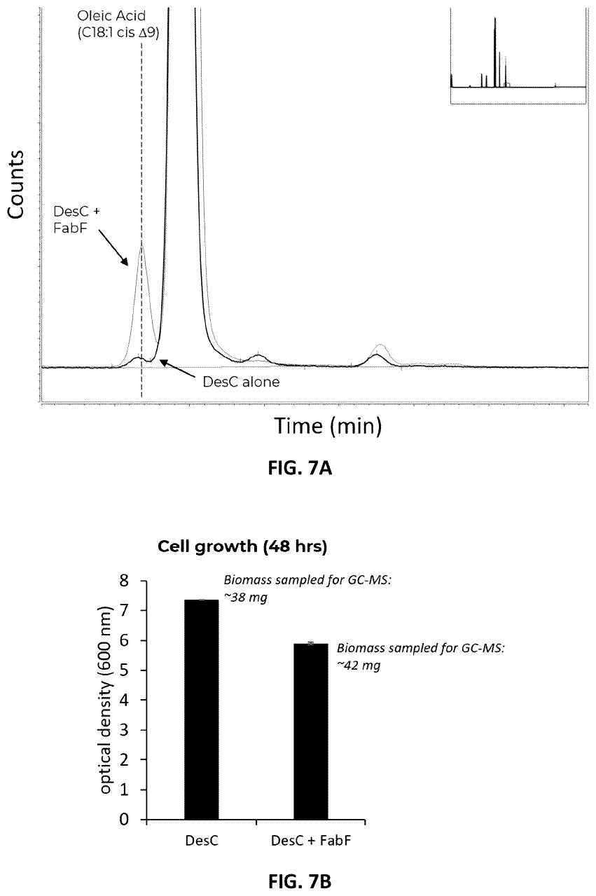

GC-MS analysis also indicates an unknown peak at 19:50 minutes that possibly indicates the production of C18:1 cis-vaccenic acid (FIGS. 4 and 5).

### Example 3—Engineering Fatty Acid Biosynthesis to Increase C18:0 Generation

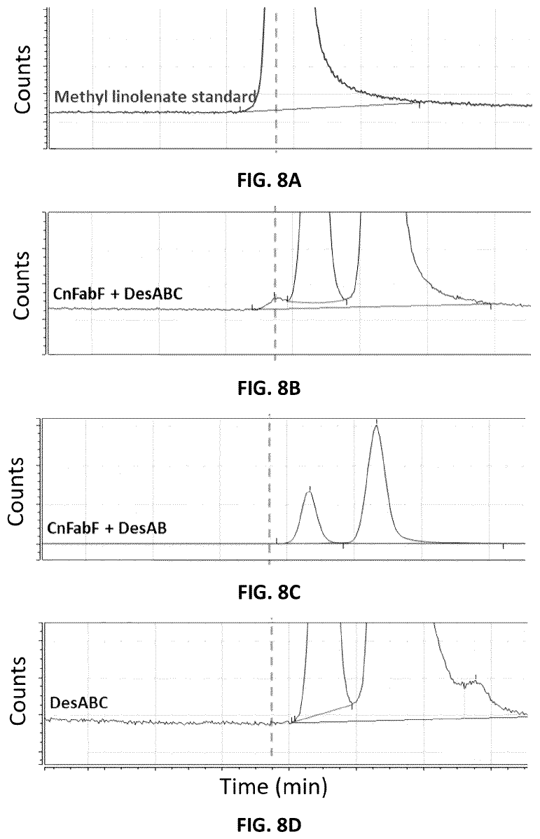

Applicant transfected temperature evolved C. necator with a Δ9-desaturase (DesC). GC-MS analysis showed a new peak in the C. necator that expressed DesC that corresponds to oleic acid and a corresponding drop in stearic acid. See FIG. 6. As a proof of concept, this showed production of a C18 unsaturated fatty acid by the engineered C. necator. C. necator was also transfected with Δ9, Δ12, and Δ15 desaturase enzymes.

### Example 4—Fab Overexpression Substantially Improved Oleic Acid Production

Overexpression of FabG or FabZ (b-reduction enzymes) has previously demonstrated increased C18:0 content in E. coli. Overexpression of FabD, FabB, or FabF (b-ketoacyl-[acyl-carrier-protein]) synthases for fatty acid initiation or elongation can shift fatty acid production towards an increase in desirable C18 fatty acid content. Moreover, the use of FabD, FabB or FabF presents the potential to elongate C16:1Δ9 rather than C16:0 β-ketoacyl-acyl carrier protein (ACP) synthase (KAS).

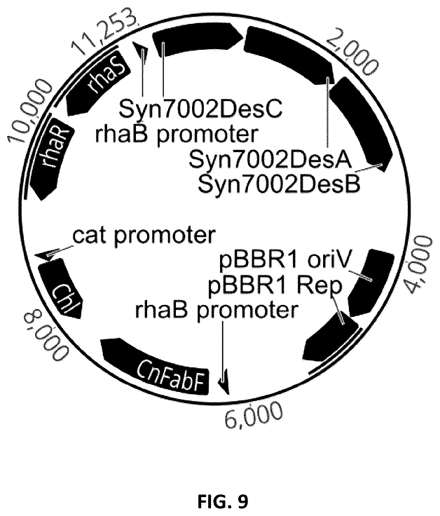

Applicant induced FabF overexpression in heat adapted strains of C. necator and analyzed the fatty acid profile of biomass at the C18 region. GC-MS analysis indicates the production of C18:1 Δ9 oleic acid production in FabF overexpressing bacteria, which was absent in DesC expressing controls alone (FIG. 7A). In addition to the production of oleic acid, FabF overexpression also demonstrated increased synthesis of C18:0 stearic acid, a crucial precursor to C18 unsaturated fatty acids.

FabF overexpression also demonstrated increased biomass confirmed through optical density analysis, indicating that Fab enzyme expression is a potential lever for improving the biosynthesis of polyunsaturated fatty acids (FIG. 7B).

### Example 5: A Linolenate Peak was Observed when CnFabF was Overexpressed Along with DesABC

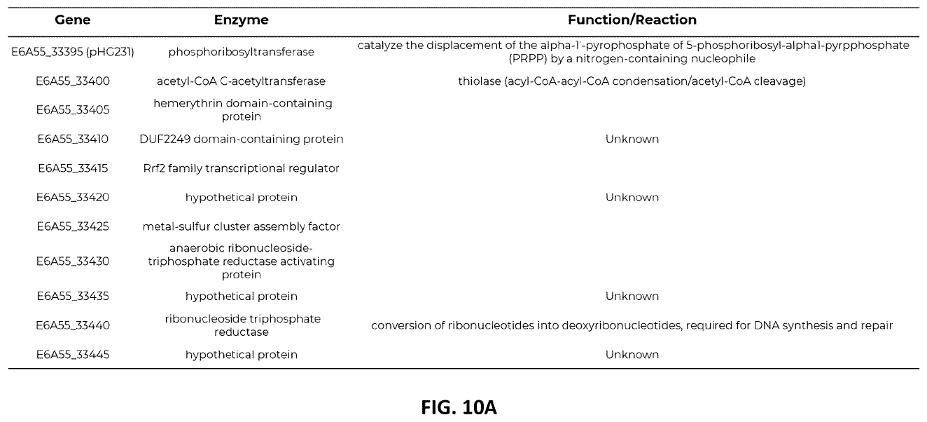

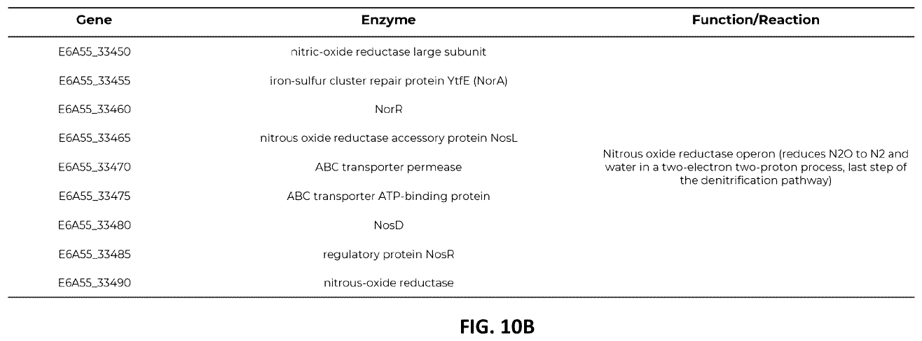

DesA and DesB desaturases from Synechococcus sp. PCC 7002 were expressed in combination with DesC in heat adapted C. necator. The plasmid utilized is shown in FIG. 9. As confirmed by the methyl linolenate standard control (FIG. 8A), the presence of methyl linolenate is results in a peak at 19.3 minutes. GC-MS analysis of CnFabF overexpression in combination with DesA, DesB and DesC demonstrated an additional peak at around 19.3 minutes (FIG. 8B), indicating linolenate production. Applicant confirmed absence of either CNFabF or DesC expression does not results in peaks corresponding to linolenate (FIGS. 8C and 8D), suggesting that the combination of FabF and DesC expression results in the synthesis of linolenic acid.

Importantly, Applicant verified that CnFabF overexpression in C. necator maintained biosynthesis of palmitic (C16), palmitoleic (C1649), and vaccenic (C18411) acids.

It should be appreciated that all combinations of the disclosed concepts are provided as being part of the inventive subject matter disclosed herein and may be employed in any combination to achieve the benefits described herein.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

1. A strain of Cupriavidus necator, comprising a transgene encoding a heterologous desaturase enzyme, wherein the strain of C. necator synthesizes at least one unsaturated C18 fatty acid that is not natively produced by C. necator.

2. The strain of C. necator of claim 1, wherein the at least one unsaturated C18 fatty acid is selected from an oleic acid, a linoleic acid, an α-linoleic acid, a γ-linolenic acid, a linolelaidic acid, an elaidic acid, a vaccenic acid, a stearidonic acid, and any combination thereof.

3. A strain of C. necator that expresses a heterologous desaturase enzyme encoded by a transgene, wherein the strain of C. necator synthesizes an increased amount of an unsaturated C16 and/or C18 fatty acid relative to a strain of C. necator that does not express the heterologous desaturase enzyme.

4. The strain of C. necator of claim 3, wherein the unsaturated fatty acid is a C16 fatty acid, a C18 fatty acid, or both a C16 fatty acid and a C18 fatty acid.

5. The strain of C. necator of claim 4, wherein the C16 fatty acid is selected from a palmitoleic acid, a sapienic acid, and any combination thereof.

6. The strain of C. necator of claim 4, wherein the C18 fatty acid is selected from an oleic acid, a linoleic acid, an α-linoleic acid, a γ-linolenic acid, a linolelaidic acid, an elaidic acid, a vaccenic acid, a stearidonic acid, and any combination thereof.

7. The strain of C. necator of claim 1, wherein the transgene is codon optimized for expression in Cupriavidus necator.

8. The strain of C. necator of claim 1, wherein the strain of C. necator is modified, optionally by adaptive laboratory evolution or genetic modification.

9. The strain of C. necator of claim 1, wherein the strain of C. necator grows autotrophically at up to 40° C.

10. The strain of C. necator of claim 8, wherein the strain of C. necator has a H₂:CO₂ uptake ratio that is lower than the H₂:CO₂ uptake ratio of a wild type or naturally occurring strain of C. necator cultured under corresponding temperatures and conditions.

11. The strain of C. necator of claim 10, wherein the strain of C. necator comprises a full or partial deletion of a gene or locus encoding a membrane-bound hydrogenase.

12. The strain of C. necator of claim 11, wherein the membrane-bound hydrogenase is hoxKGXZ.

13. The strain of C. necator of claim 1, wherein the strain of C. necator produces about 40%, about 50%, about 60%, about 70%, about 80%, about 90%, or about 95% by weight of protein.

14. The strain of C. necator of claim 1, wherein the heterologous desaturase enzyme is selected from a Δ9-desaturase, a Δ12-desaturase, a Δ15-desaturase, a Δ6-desaturase, a Δ5-desaturase, and a Δ4-desaturase.

15. The strain of C. necator of claim 1, wherein the heterologous desaturase enzyme is from a cyanobacteria or a eukaryote.

16. The strain of C. necator of claim 15, wherein the cyanobacteria is a Synechococcus species (sp).

17. The strain of C. necator of claim 1, wherein the strain of C. necator further comprises a second transgene encoding a second heterologous desaturase enzyme.

18. The strain of C. necator of claim 17, wherein the second heterologous desaturase enzyme is selected from a Δ9-desaturase, a Δ12-desaturase, a Δ15-desaturase, a Δ6-desaturase, a Δ5-desaturase, and a Δ4-desaturase.

19. The strain of C. necator of claim 17, wherein the second heterologous desaturase enzyme is from a cyanobacteria or a eukaryote.

20. The strain of C. necator of claim 17, wherein the strain of C. necator further comprises a third transgene encoding a third heterologous desaturase enzyme.
